(19) United States
(12) Patent Application Publication
(10) Pub. No.: US 2005/0106707 A1
(43) Pub. Date: May 19, 2005

(54) Automatic apparatus for synthesis of small organic molecules and synthesis method using same

(76) Inventors: Jean Neimark, Strasbourg (FR); Jean-Michel Henlin, Surennes (FR); Jean-Luc Fauchere, Saint-Cloud (FR); Gisele Fauchere, Saint-Cloud (FR)

Correspondence Address: YOUNG & THOMPSON, 745 SOUTH 23RD STREET, 2ND FLOOR, ARLINGTON, VA, 22202, US

(21) Appl. No.: 10/488,993

(22) PCT Filed: Sep. 9, 2002
(86) PCT No.: PCT/FR02/03059

(30) Foreign Application Priority Data
Sep. 10, 2001 (FR) 01/11694

(51) Int. Cl. B32B 5/02; G01N 21/00; C12M 1/34

(52) U.S. Cl. 435/287.1; 422/82; 435/7.1; 436/518

(57) ABSTRACT

An automatic apparatus for synthesis of organic molecules, in accordance with combinative or parallel synthesis protocols. The apparatus comprises: several synthesis modules including each five reactors temperature-controlled by heating and cooling elements, a container forming a secondary mixing chamber whose capacity corresponds to at least the sum of capacities of the reactors of a module, being associated with each module and an additional container, forming a main mixing chamber whose capacity corresponds to at least the sum of capacities of the various secondary mixing chambers, and at least a circuit for transferring the contents of the reactors to one or more containers or reactors, and at least a supply or discharge circuit connected to the different inlets/outlets of the reactors and of the containers and, finally, a ramified system for managing and controlling the flow and distribution of fluids in the circuits and the temperature and stirring in the chambers.

The present invention relates to the field of the production by synthesis of organic molecules, preferably small organic molecules in a large number by simultaneous parallel syntheses, and has for its object an automatic apparatus for the synthesis of organic molecules, according to combinative or parallel synthesis protocols, preferably in solid phase.

The combinatory synthesis forms a part of the arsenal of the pharmaceutical industry to contribute to the discovery of new active organic molecules, in particular new medications. It has for its object the rapid preparation of numerous products adapted for pharmacological screening. To do this, it accelerates the process of discovery of new chemical entities and increases their molecular diversity.

Moreover, those skilled in the art know the advantages of synthesis in solid phase relative to synthesis in solution, which have been described for polypeptides and polynucleotids and for the other structural classes of organic molecules. It is a matter in this instance of easy intermediate purification of products by washing and filtration, and of the possible automation of the synthetic process.

In FR-A-2 582 655, there has already been disclosed a multi-synthesizer of peptides in solid phase with semi-automatic operation, having more than two simultaneous synthesis paths and using the synthesis technique in solid phase developed by B. MERRIFIELD.

This apparatus was the subject of additional developments and improvements, particularly as to automation and continuous production without manual intervention, to lead to an automatic simultaneous synthesis of several identical or different peptides in solid phase described and illustrated in FR-A-2 664 602.

Given the advantages of each of the two synthesis techniques described above, different developments have been carried out to employ combinative synthesis methods in solid phase. Such a method permits preparing mixtures of products thanks to mixing and redistribution operations of the intermediate products. This sharing of the intermediate synthesis products, with one or several repetitions in the course of a complete synthesis protocol, is preferably used to carry out on said mixed intermediate products, synthesis operations or the like forming a part of synthesis protocols of each of the products to be obtained.

Such a combinative process multiplies at each step the number of products obtained by a number at least equal to the number of reactors used. Thus, five steps of coupling of 20 synthons preliminarily mixed, produce an expected number of 64 millions of hexamer products.

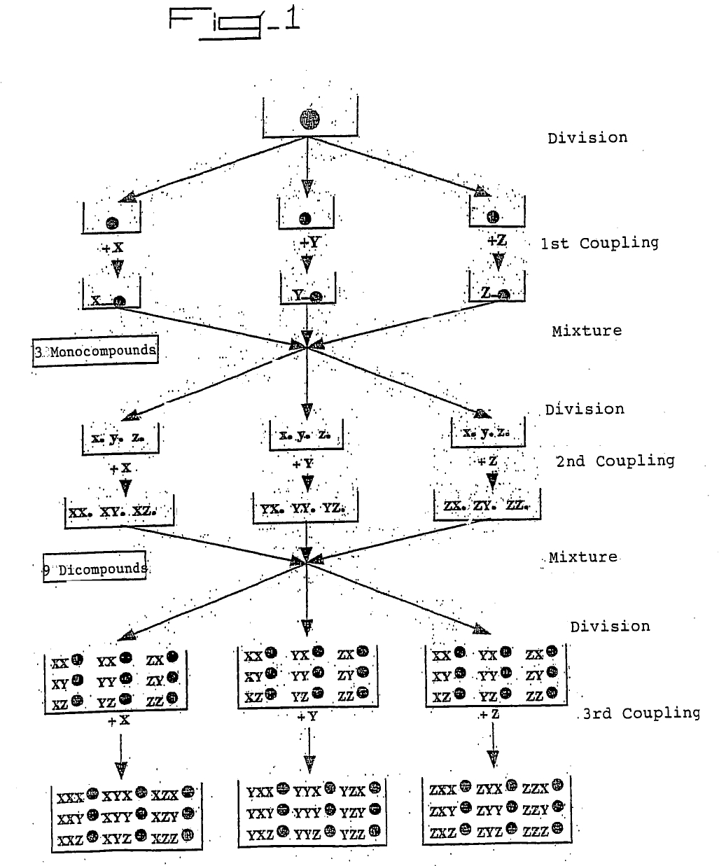

FIG. 1 of the accompanying drawings shows graphically the principle of combinative synthesis on a solid support with the help of an example using three compounds or chemical elements of different bases, commonly called synthons, to produce twenty-seven different compounds each formed from three synthons. The illustrated example permits accordingly arriving at three libraries of nine tri-compounds each obtained by means of nine synthesis operations.

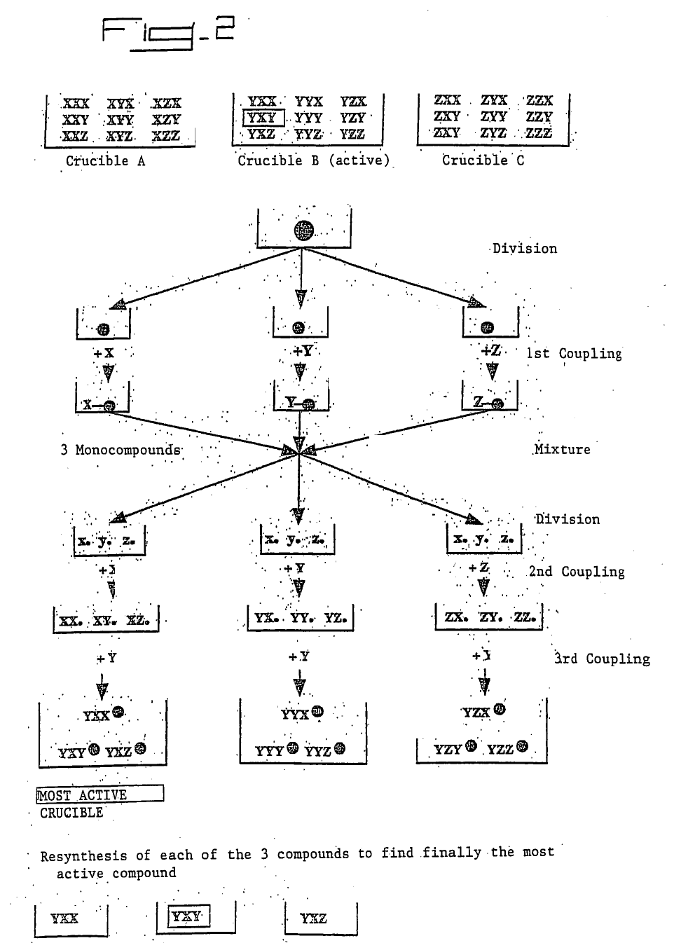

FIG. 2 also shows by way of example, an embodiment of a deconvolution process permitting seeking, among the three libraries of tri-compounds precedingly obtained, the tri-compound which is biologically the most active. After identification of the receptacle or crucible containing the most active product, there is carried out a re-synthesis of the three tri-compounds of said crucible to determine finally the most active tri-compound.

An automation of such a combinative synthesis technique in solid phase, at constant temperature and in open reactors whose internal volume is accessible, has already been proposed (cf: Boutin, J. A. & Fauchère, J. L. (1996) “Second-generation robotic synthesizer for peptide, pseudopeptide and non-peptide libraries”, Proceedings of the International Symposium on Laboratory Automation and Robotics 1995, Zymark Corp., Hopkinton, MA, USA. Pp. 197-210/cf: Zuckermann et al.: “Design, construction and application of a fully automated, equimolar peptide mixture synthesizer”, Int. J. Peptide Protein Res. 40 (1992) 497).

Moreover, different automata for organic parallel synthesis of individual products in solid phase or in solution are at present available commercially, as for example those known by the term MYRIAD of the METTLER-TOLEDO company, these automata however not permitting practicing combinative synthesis techniques, comprising at least one step of mixing and redistribution of the intermediate products.

Moreover, the known apparatus mentioned above are generally dedicated, because of their own construction and structure, to a type of synthesis technique, comprising portions, pieces or mechanical members in movement such as arms moved in translation and/or in rotation to carry out at least certain of the transfers of substances, particularly liquids, and are limited as to the possibilities of thermal conditions applicable to the reactions to be carried out and/or not permitting an extension of capacity by addition of identical modules, without modification of the entire structure.

The problem posed by the present invention consists particularly in overcoming at least certain of the mentioned drawbacks and in surmounting at least certain of the limitations of the existing apparatus.

The principal object of the present invention is to provide automatic apparatus for the synthesis of organic molecules by application of combinative synthesis techniques over a wide range of temperatures and under controlled atmosphere, comprising no movable member or piece in the chamber of said apparatus in the course of the operative phases and permitting easy extension of the capacity and supple and flexible programming of different combinative synthesis protocols, as well as an easy reconfiguration for utilization in parallel synthesis.

To this end, the present invention has for its object an automatic apparatus for the synthesis of organic molecules, particularly in solid phase, according to combinative or parallel synthesis protocols, principally constituted, on the one hand, by several synthesis modules each comprising between three and ten, preferably five, reactors each formed by a tubular body delimiting, in cooperation with an upper injection and expansion plug and a lower removable plug for controlled withdrawal and emptying, a normally closed sealed reaction chamber controlled as to temperature by heating and cooling means and provided with agitation means for the reaction medium by bubbling and/or by means of a mechanical member, a container forming a secondary mixing chamber and whose capacity corresponds at least to the sum of the capacities of the reactors of a module, being associated with each module and a supplemental container being provided, forming a principal mixing chamber whose capacity corresponds at least to the sum of the capacities of the different secondary mixing chambers, on the other hand by at least one circuit for the transfer of the contents of the reactors toward the container associated with the synthesis module in question and/or toward the container and the controlled distribution of the container of one or more containers between the reactors of the associated module and/or of the capacity of the container between the containers or reactors of one, several or all the modules of the apparatus, as well as at least one supply or evacuation circuit connected particularly to the different inputs/outputs of the reactors and the containers, the circuits permitting the circulation of fluid or fluids under the action of a propulsive inert or neutral gas and being formed by conduits or portions of conduits interconnected to each other and connecting said reactors and containers to each other and to reservoirs of solutions and to lines for expansion, suction and injection of propulsive or bubbling gas, these connections being established temporarily by means of one-way valves or units of multi-way and monobloc valves constituting programmable junctions for configuring said circuits or control and management members of the inputs/outputs of the reactors and containers and, finally, by a branched supervisory system of control and management of the circulation and distribution of fluids in the mentioned circuits and of the temperature and agitation in the chambers of the reactors, comprising particularly a computer unit associated with electronic interfacing and multiplexing circuits particularly for the control of the valves and valve units, heating means and cooling means of the reaction chambers and, as the case may be, of driving the mechanical agitation members, and integrating interfaces of dialog with and programming by the user.

The invention will be better understood from the following description, which relates to a preferred embodiment, given by way of non-limiting example, and explained with reference to the accompanying schematic drawings, in which:

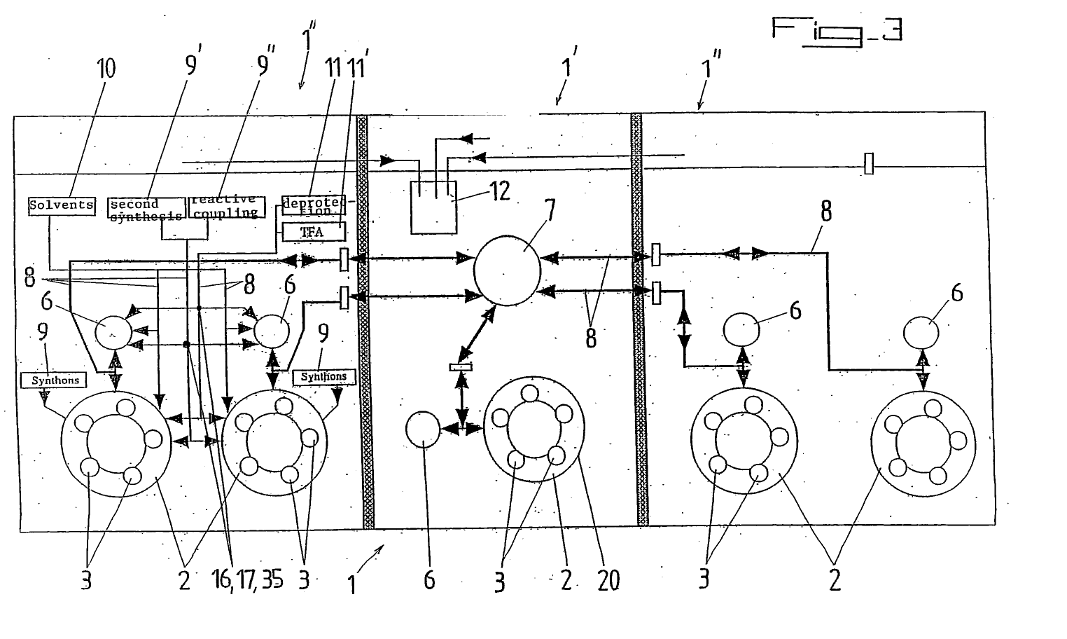

FIG. 3 is a partial schematic representation of automatic apparatus showing the arrangement of its modular structure and the principal circuits for circulation and fluid exchange, in particular for one of the sub-units;

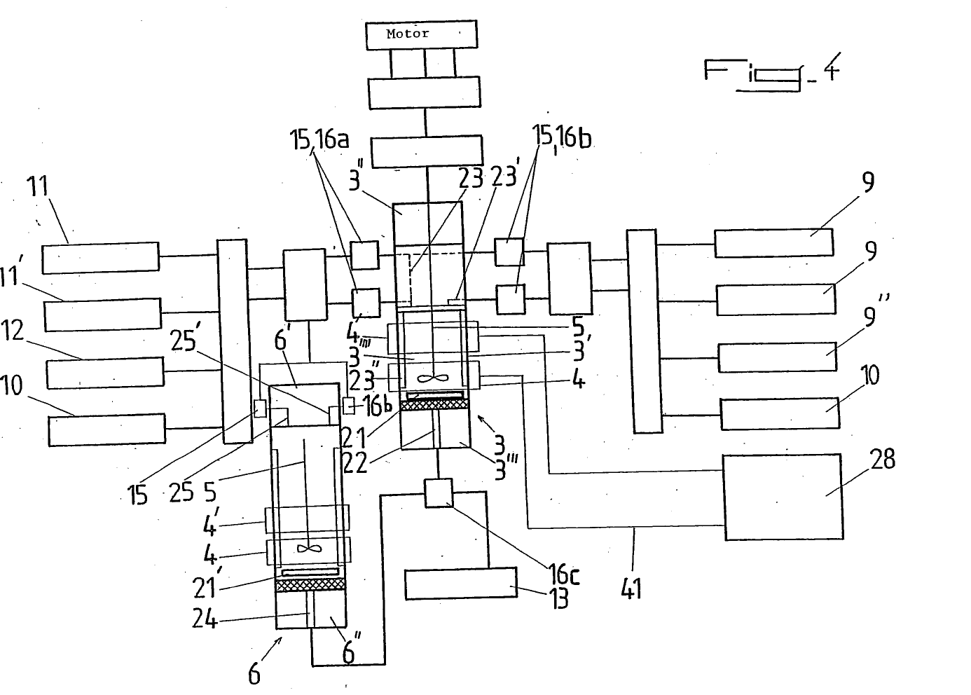

FIG. 4 is a partial synoptic representation showing symbolically the circuits connected to the reactors and to the secondary mixing chambers (for reasons of understanding and simplification of the representation a single reactor and a single secondary mixing chamber are shown);

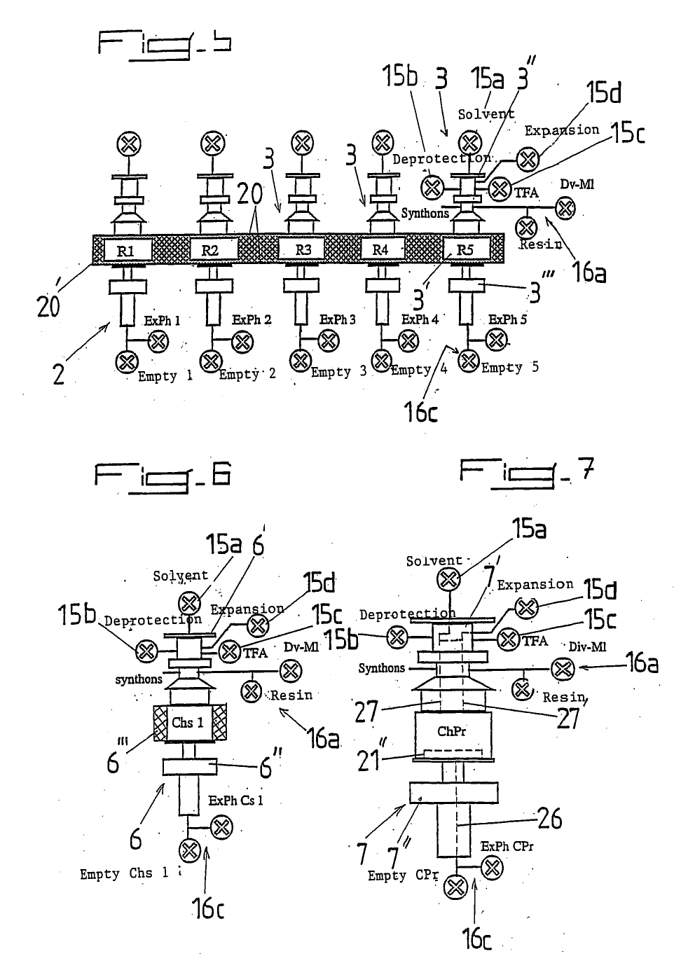

FIG. 5 is a schematic representation of a synthesis module forming a part of the apparatus according to the invention, also showing the different inputs/outputs at the level of the upper and lower plugs of said reactors (for reasons of simplification of the representation only the reactor R5 takes account of all said inputs/outputs);

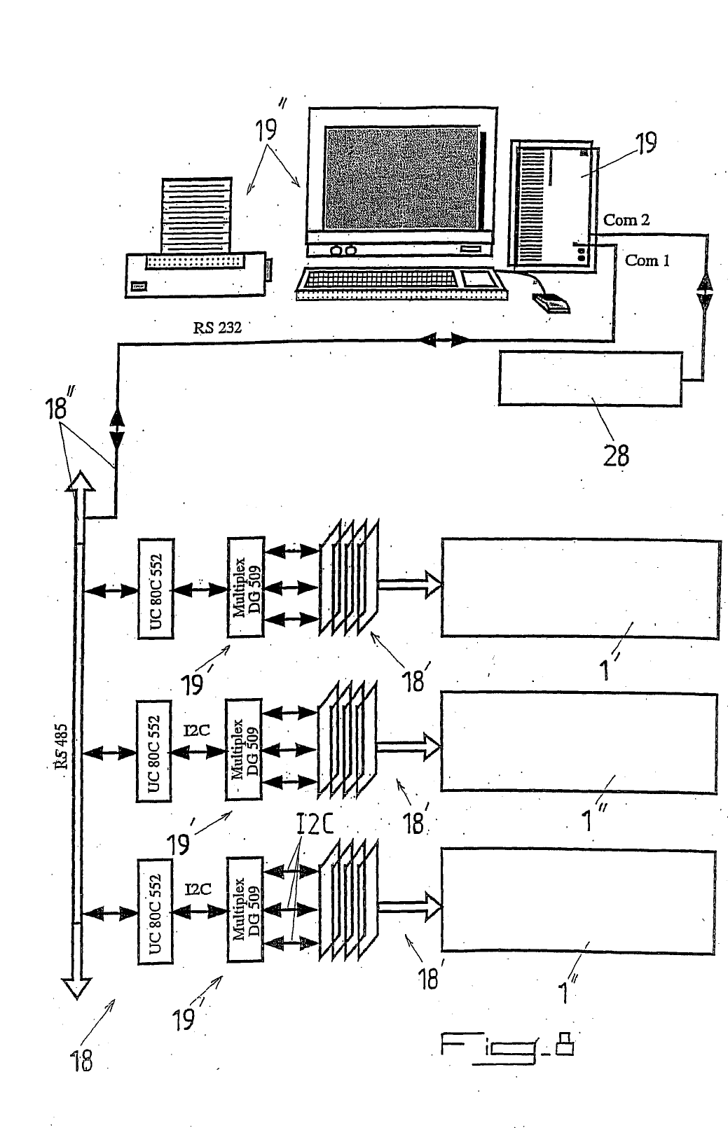

FIG. 6 is a schematic representation of a container forming a secondary mixing chamber, showing particularly its different inputs/outputs at the level of the upper and lower plugs;

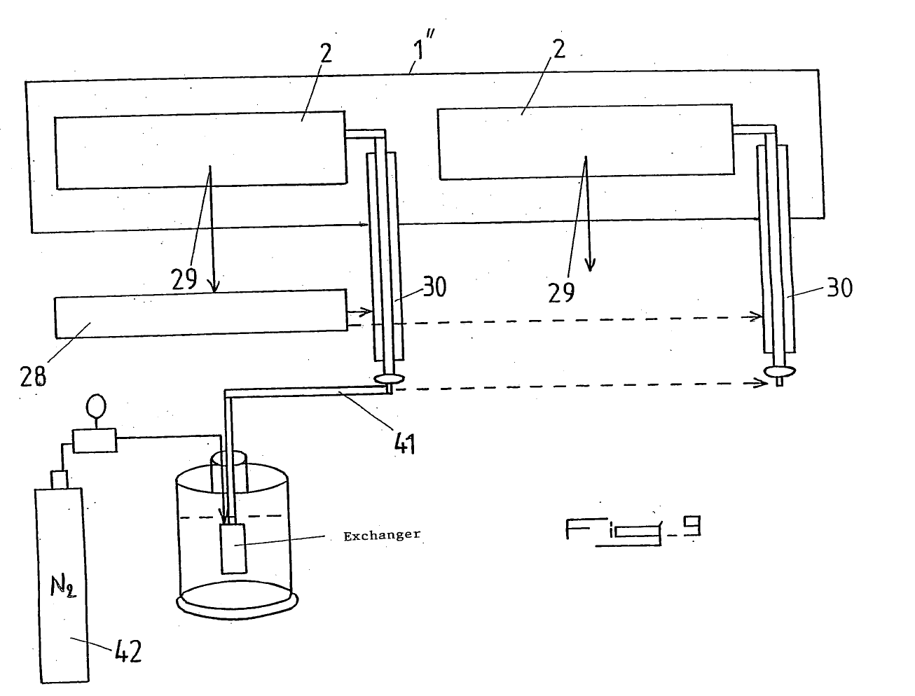

FIG. 7 is a schematic representation of a container forming a principal mixing chamber, showing particularly its different inputs/outputs at the level of its upper and lower plugs;

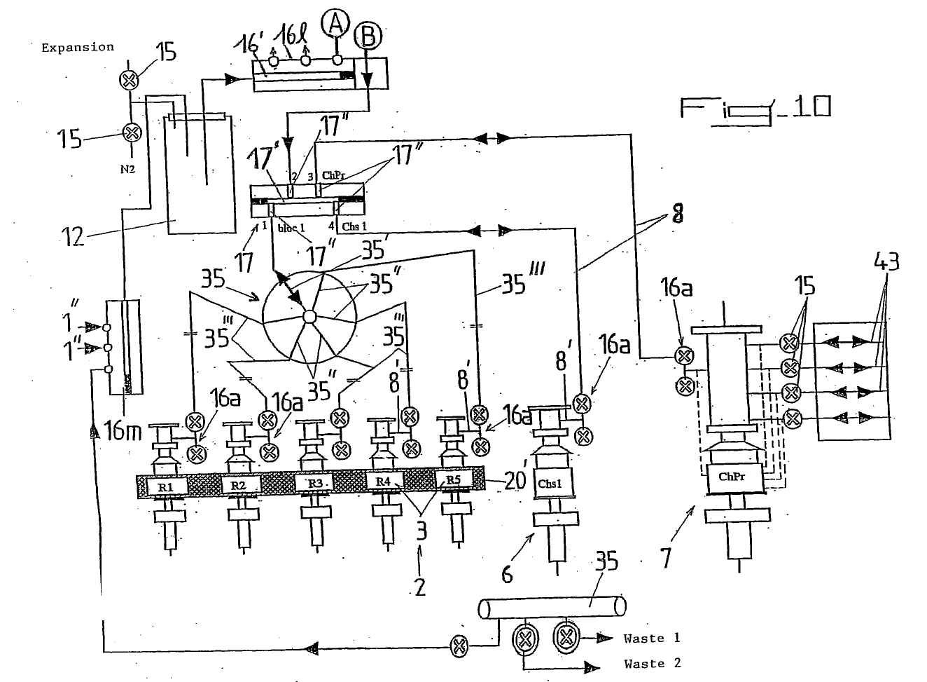

FIG. 8 is a schematic representation of the branched supervisory system forming a part of the automatic apparatus according to the invention;

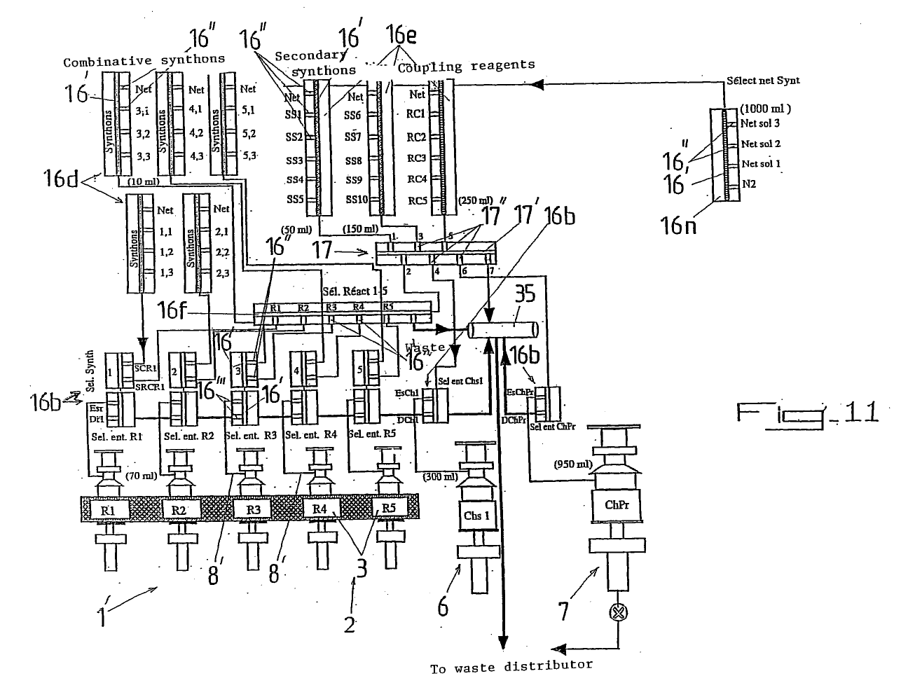

FIG. 9 is a schematic representation of a device for regulating the temperature of a sub-unit of the apparatus according to the invention;

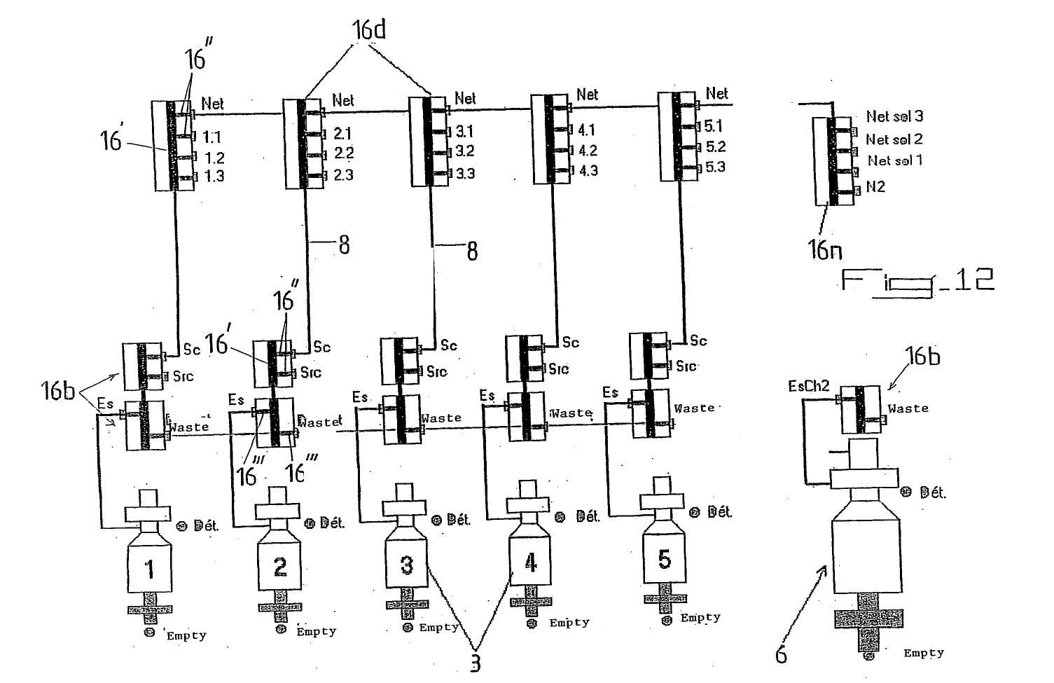

FIG. 10 is a fluid diagram of the circuit for distribution/partitioning/mixing forming a portion of the sub-unit comprising a synthesis module and the principal mixing chamber;

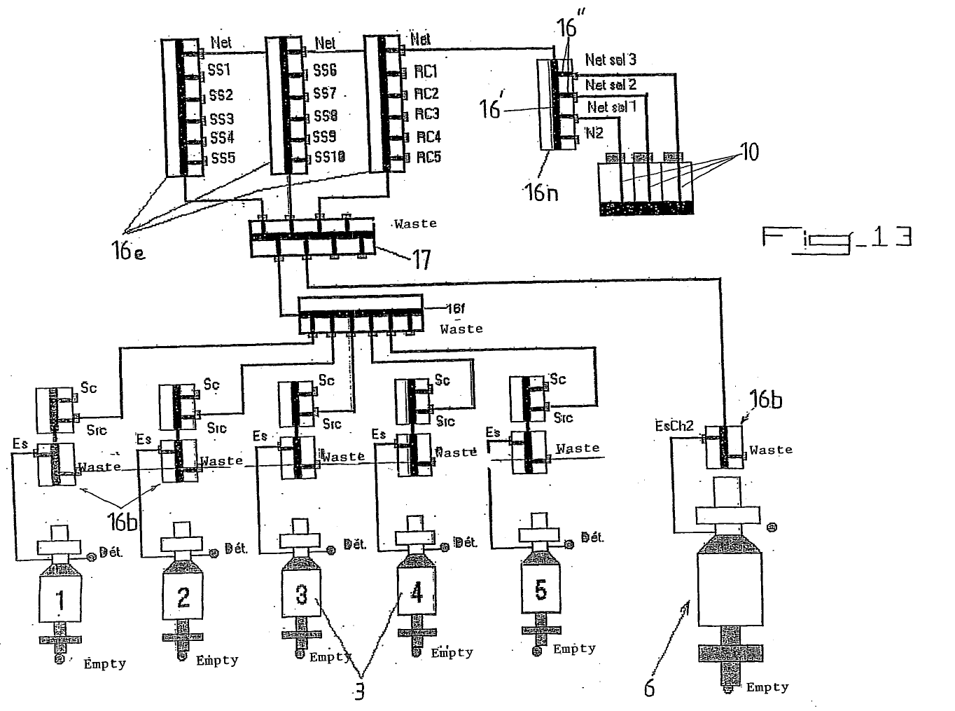

FIG. 11 is a fluid diagram of the distribution circuit of the synthons of at least a portion of the coupling reactors of the sub-unit comprising a synthesis module and the principal mixing chamber;

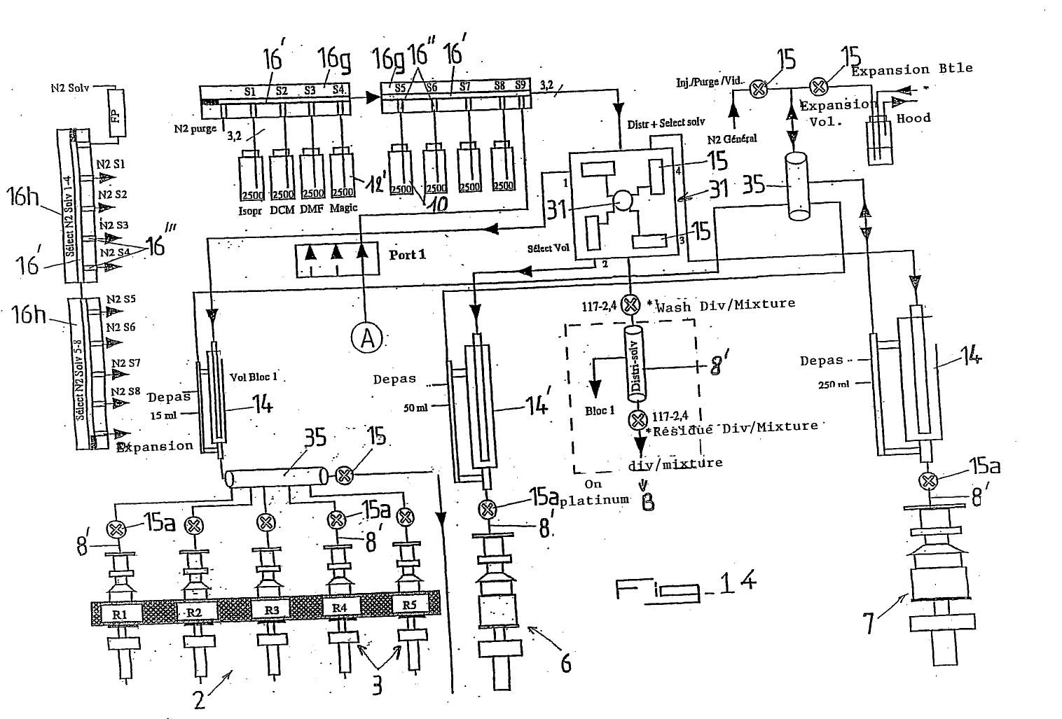

FIG. 12 is a fluid diagram showing the sub-circuit for supply of combinative synthons for a synthesis module and an associated secondary mixing chamber;

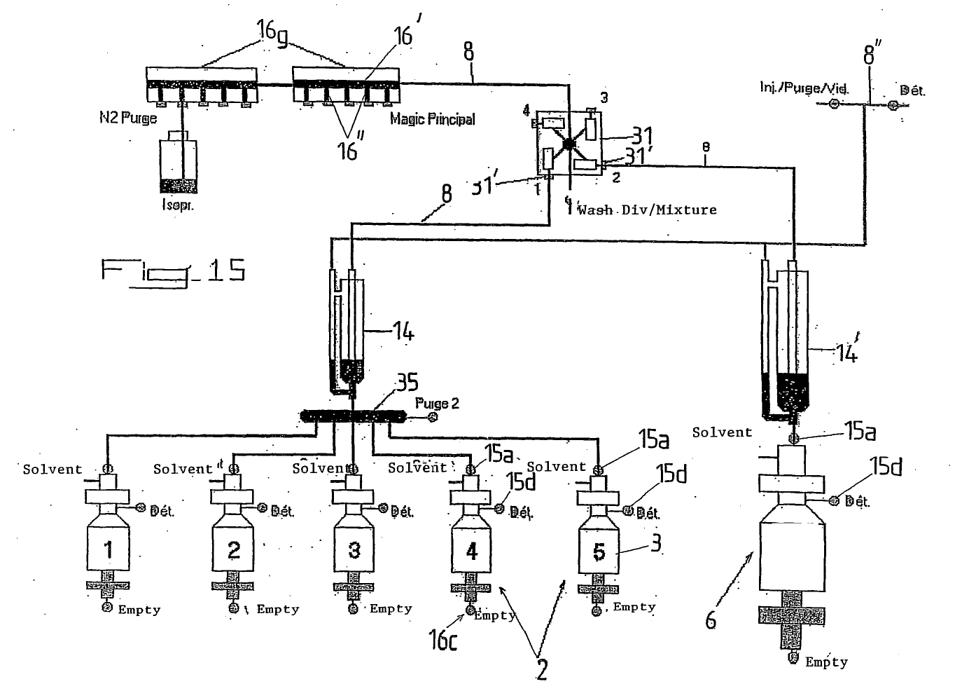

FIG. 13 is a fluid diagram showing the sub-circuit for supply of secondary or common synthons and of coupling reagents for a synthesis module and an associated secondary mixing chamber;

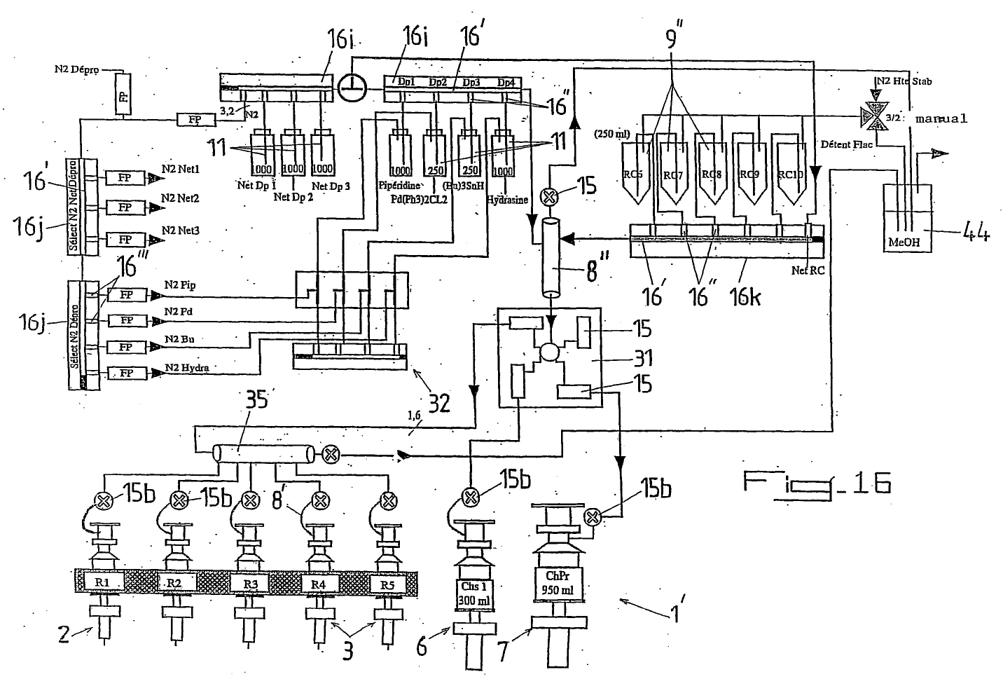

FIG. 14 is a fluid diagram of the distribution circuit for washing solvents, the distribution circuit for solvents for washing, cleaning and rinsing the sub-unit comprising a synthesis module and the principal mixing chamber;

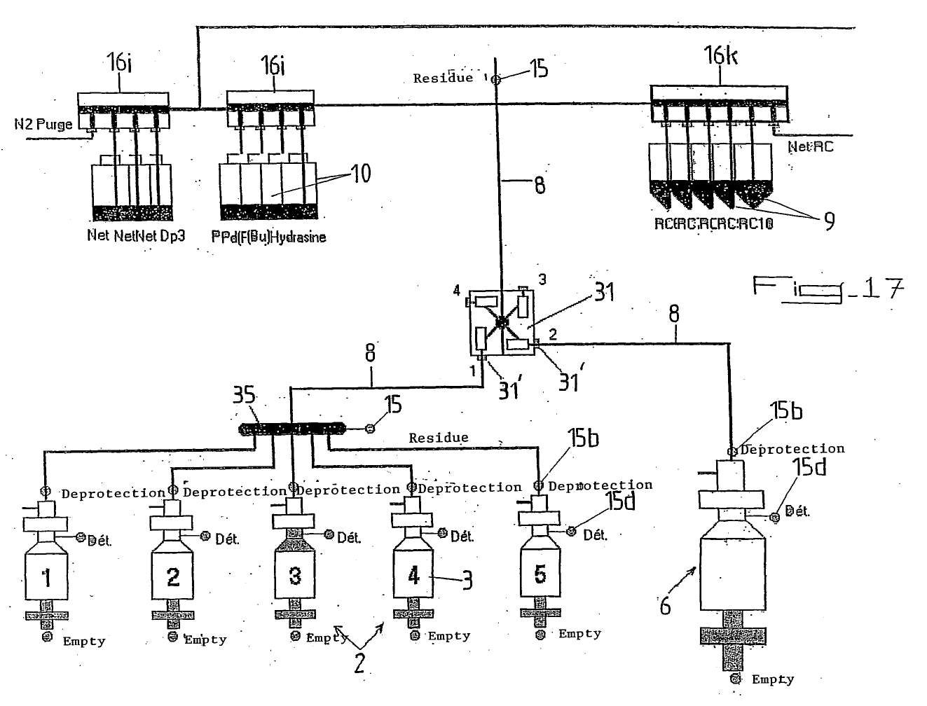

FIG. 15 is a partial fluid diagram of the distribution circuit shown in FIG. 13 during an operation of washing the reactors of a module and of the associated secondary mixing chamber;

FIG. 16 is a fluid diagram of the distribution circuit for general de-protection reagents and a secondary circuit for distribution of coupling reagents, for the sub-unit comprising a synthesis module and the principal mixing chamber;

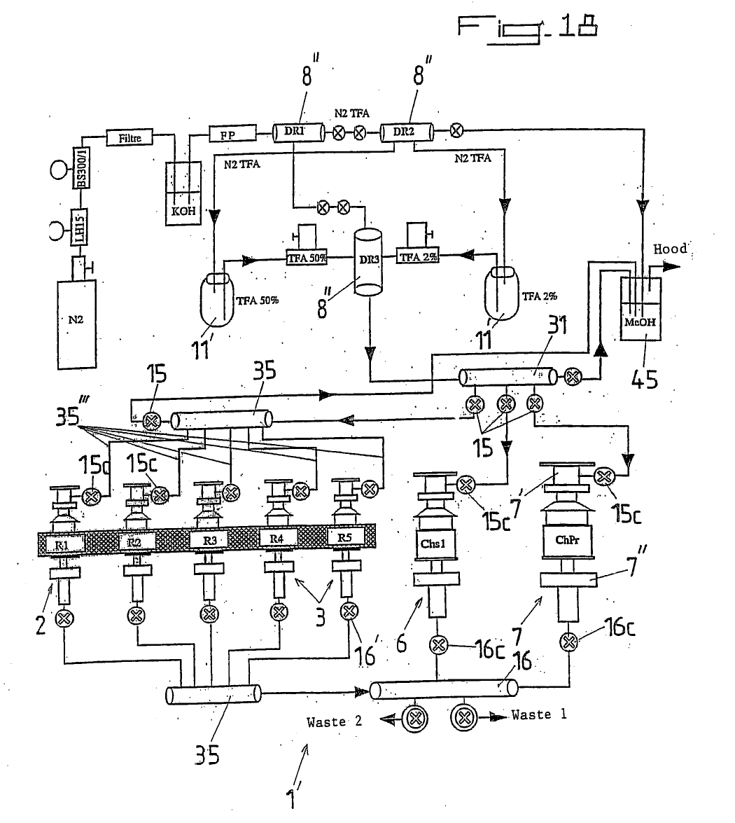

FIG. 17 is a partial fluid diagram of the distribution circuit for reagents for general de-protection feeding a synthesis module and the associated secondary mixing chamber;

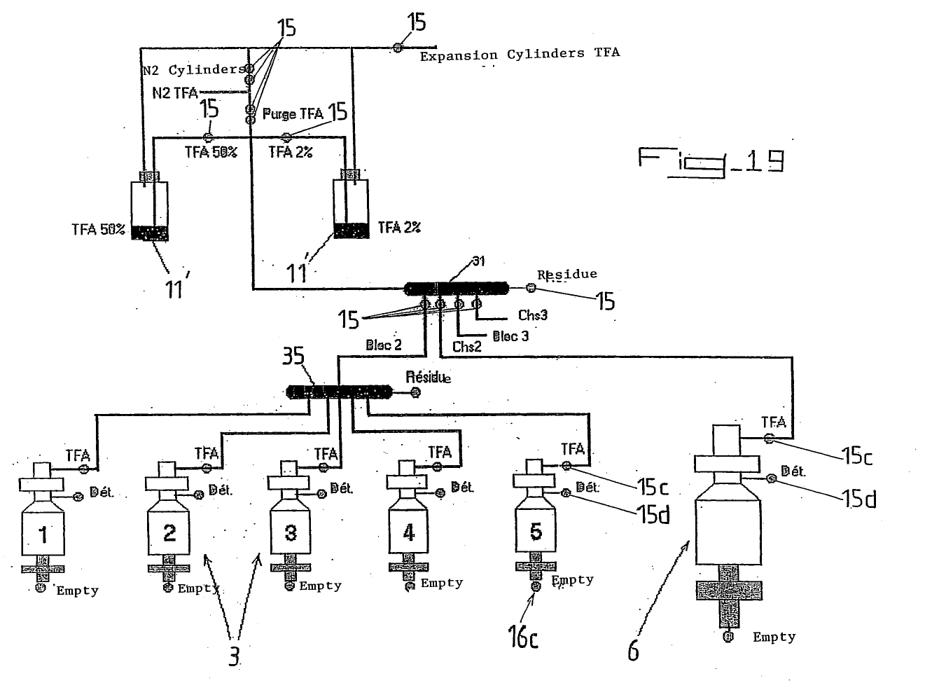

FIG. 18 is a fluid diagram of the distribution circuit for reagents for TFA de-protection of the sub-unit enclosing a synthesis module and the principal mixing chamber, this figure also showing the evacuation circuit for waste and for emptying this sub-unit;

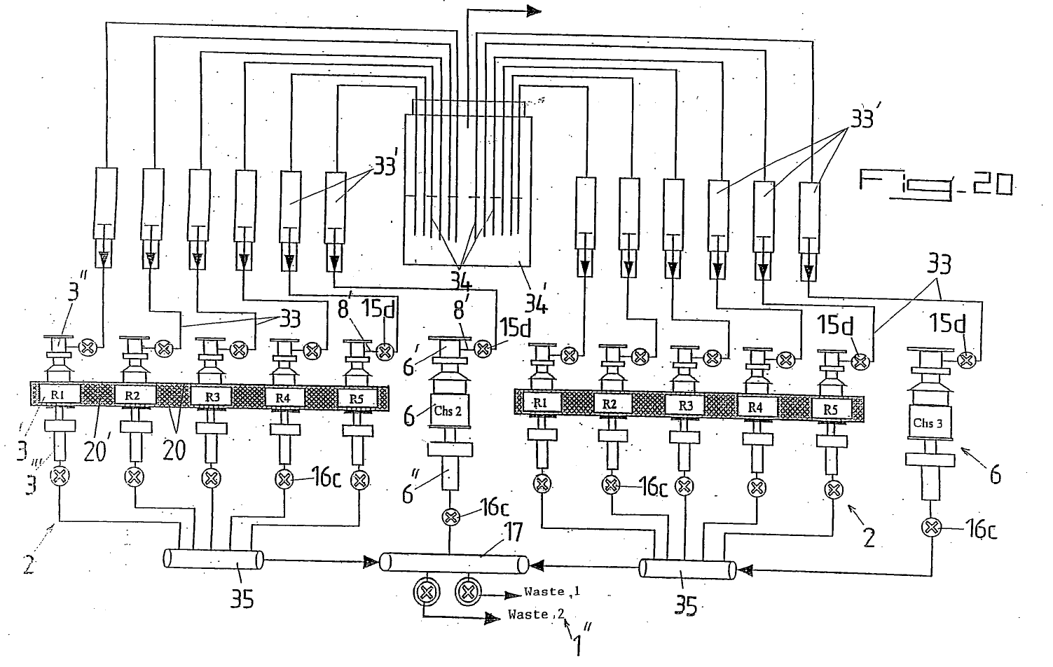

FIG. 19 is a partial fluid diagram of the distribution circuit for TFA de-protection reagents supplying a module and the associated secondary mixing chamber;

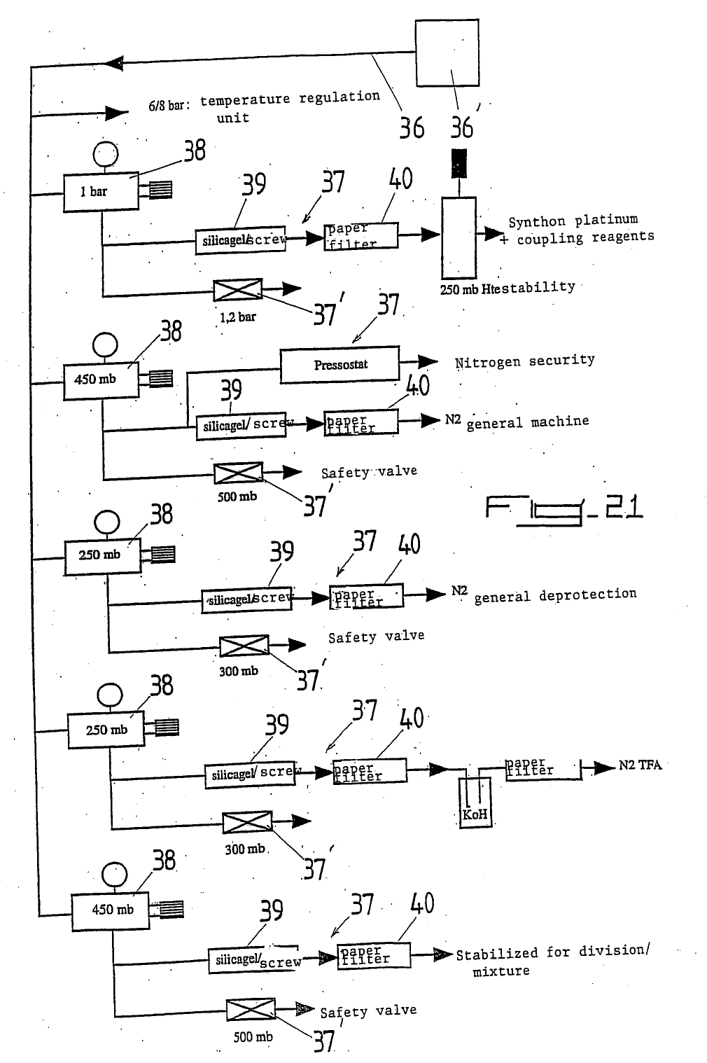

FIG. 20 is a fluid diagram of the expansion circuit and of the evacuation circuit for waste and for emptying the reactors and secondary mixing containers forming a part of a sub-unit comprising two synthesis modules;

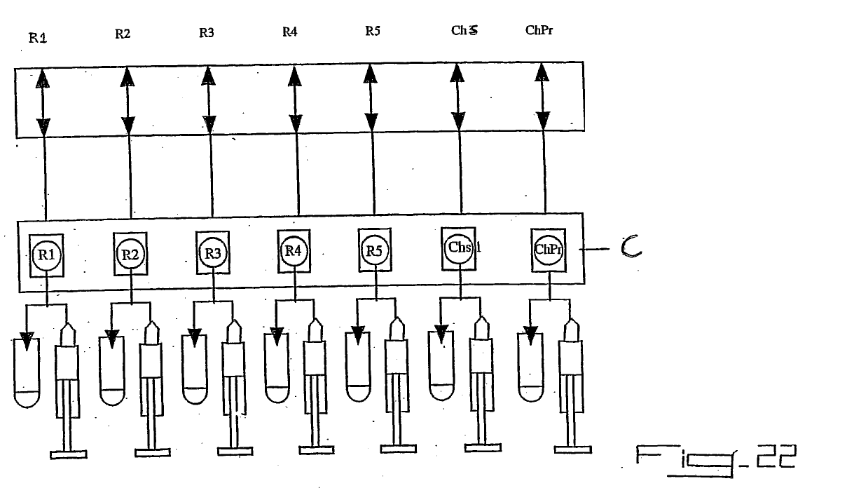

FIG. 21 is a fluid diagram of the circuit for distribution and injection of a propulsive gas, in this case N₂, supplying the assembly of the reservoirs, circuits, reactors and container for the apparatus according to the invention, to transfer fluids and, as the case may be, for bubbling;

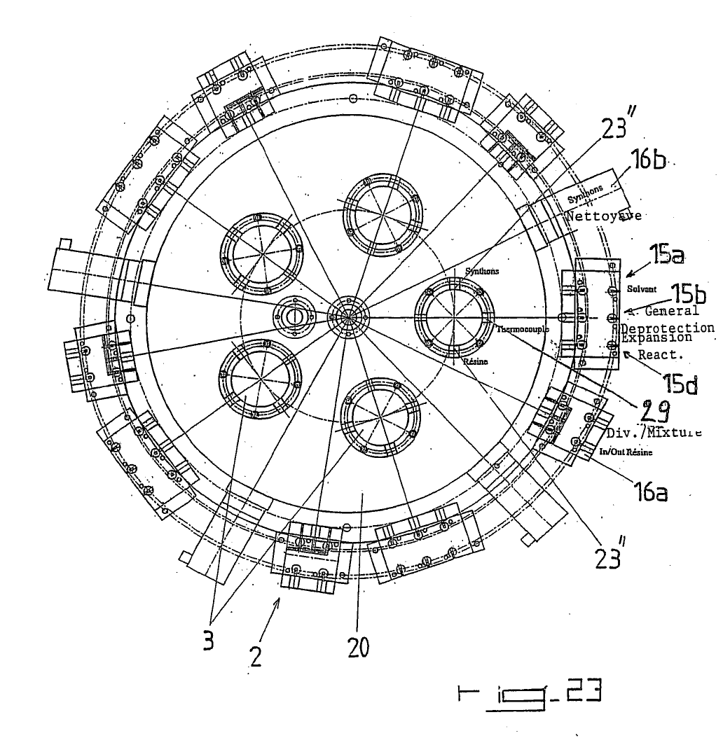

FIG. 22 shows schematically the structure of the injection lines for resin associated with a sub-unit enclosing a synthesis module and the container forming the principal mixing chamber;

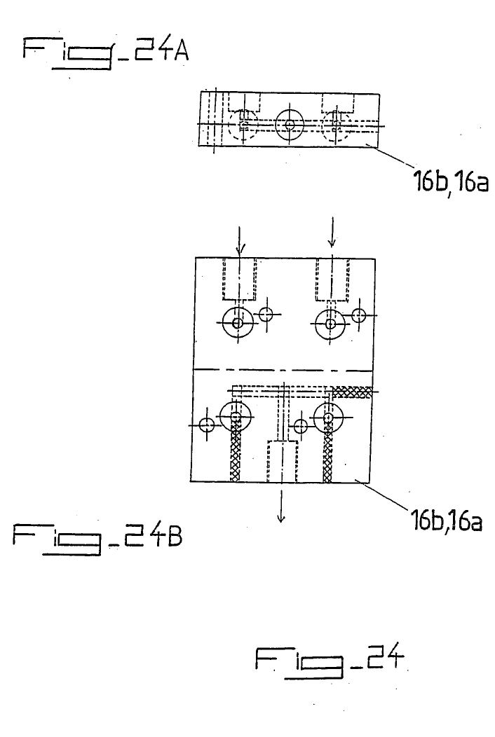

FIG. 23 is a top plan view of a synthesis module of five reactors and units of management valves for the inputs/outputs at the level of the upper plugs of said reactors, according to a practical and non-limiting embodiment of the invention;

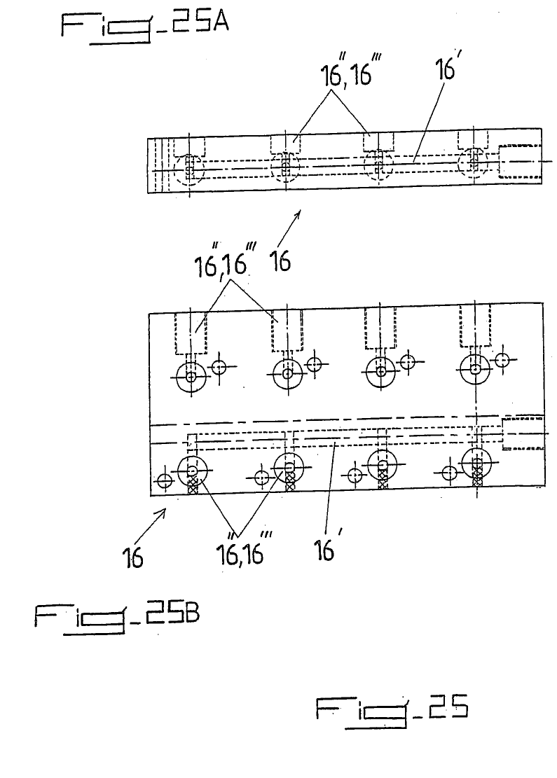

FIGS. 24A and 24B are view in side elevation and from above of a multi-path valve unit for management of the inlets and outlets of a communication channel (injection/evacuation) of a reactor;

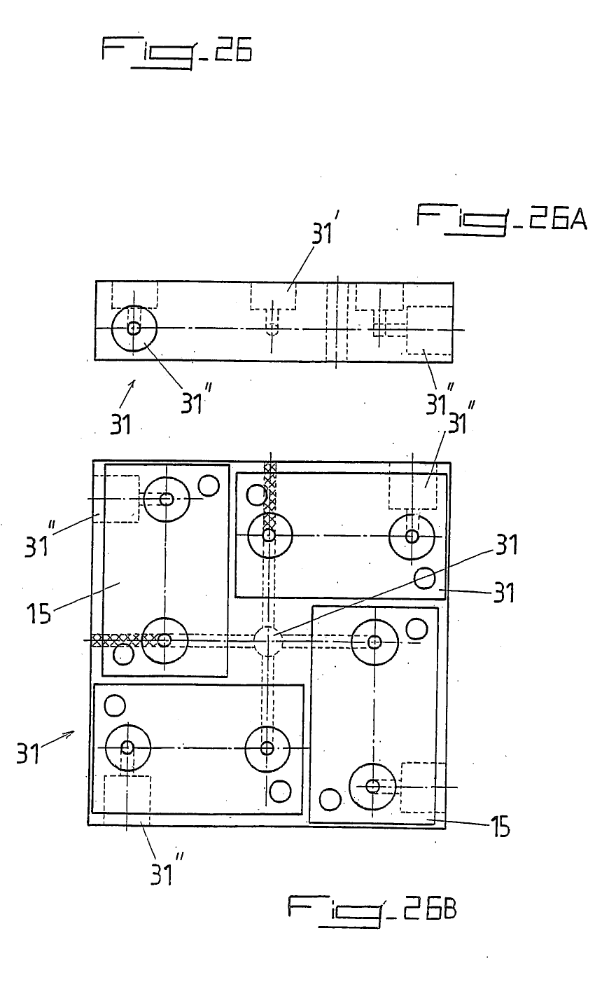

FIGS. 25A and 25B are views in side elevation and from above of a multi-path valve unit for selection or distribution forming a portion of the apparatus according to the invention;

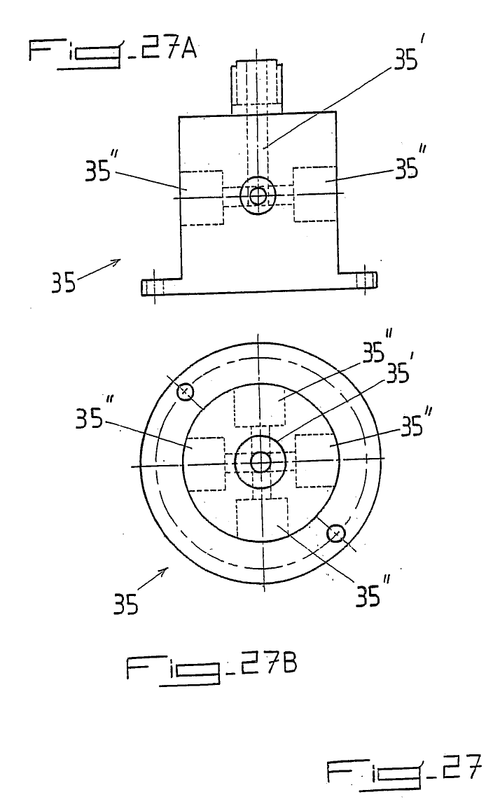

FIGS. 26A and 26B are views in side elevation and from above of a radial distributor block forming a part of the apparatus according to the invention;

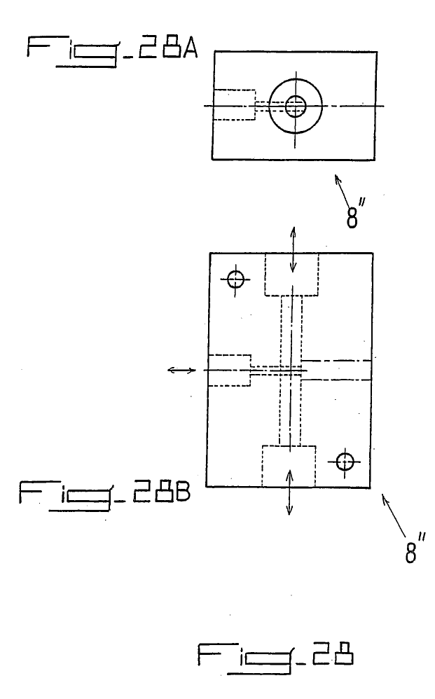

FIGS. 27A and 27B are views in side elevation and from above of an equi-molar divider with four paths, or of a four-path collector, this as a function of its mounting, forming a part of the apparatus according to the invention;

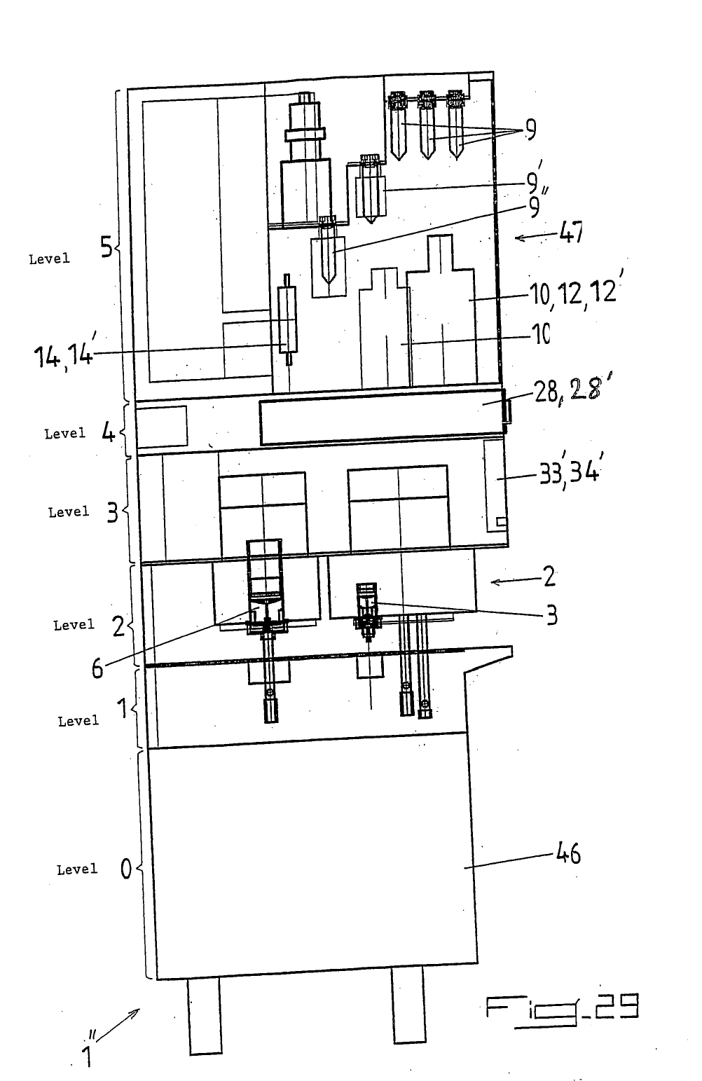

FIGS. 28A and 28B are views in side elevation and from above of an equi-molar divider with two paths, or of a two-path collector, this as a function of its mounting, forming a portion of the apparatus according to the invention;

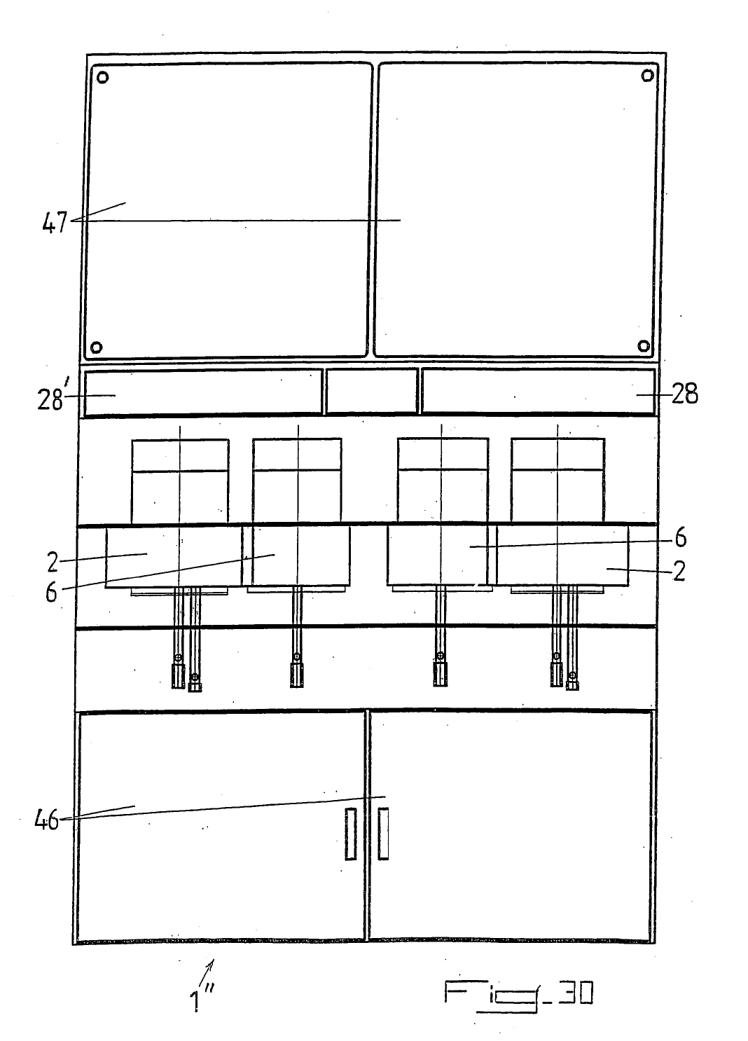

FIG. 29 is a side elevational view partly transparent of a current sub-unit of an automatic synthesis apparatus according to a practical embodiment of the invention;

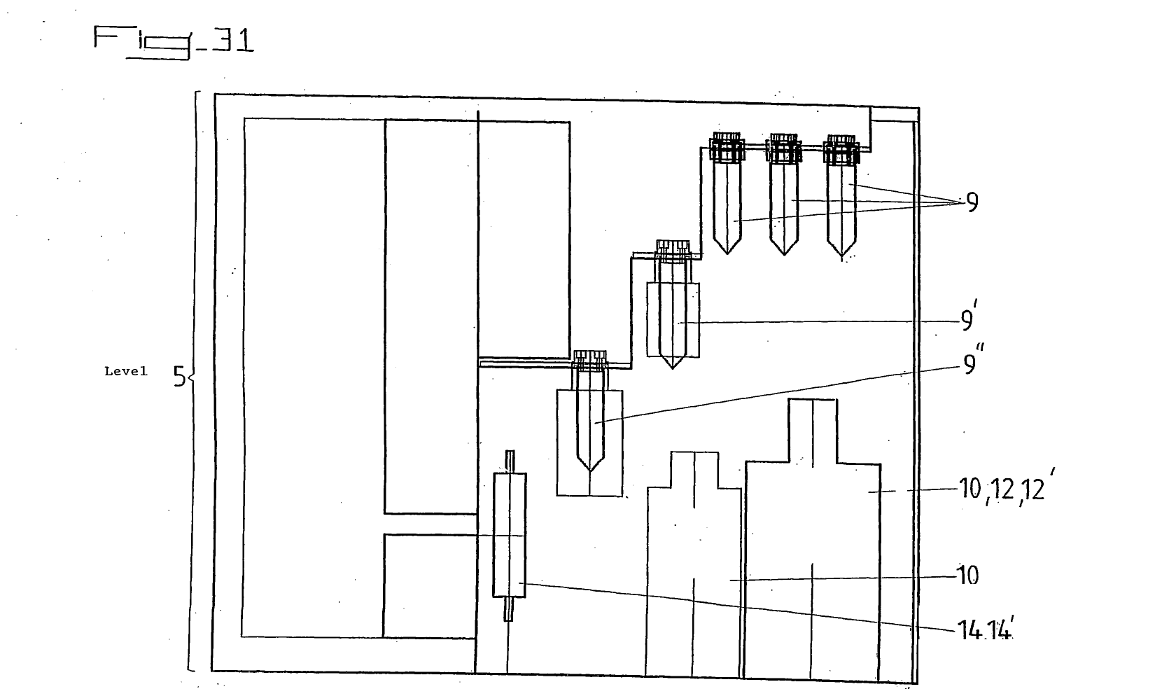

FIG. 30 is a front elevational view of the sub-unit shown in FIG. 29, and

FIG. 31 is a side elevational transparent view on a different scale, of the upper stage of the sub-unit shown in FIG. 29.

FIGS. 3 to 28 each show, in a more or less schematic form, a portion only of a constituent element of the automatic synthesis apparatus 1 for molecules, for the synthesis of organic molecules and which permits, by association, having a complete representation of all the constituent parts of said apparatus 1. In particular, FIGS. 10 to 20 each show only one circuit for a given fluid circulation for a modular 2 or a sub-unit 1′, 1″ of the apparatus 1, the other modules or sub-units having a similar circuit.

This apparatus 1 is principally constituted, on the one hand, by several synthesis modules 2 each comprising between three and ten, preferably five, reactors 3 each formed by a tubular body 3′ delimiting, in cooperation with an upper injection and expansion plug 3″ and a lower removable plug 3′″ for controlled withdrawal and emptying, a reaction chamber 3″″ normally closed in a sealed manner and controlled as to temperature by heating and cooling means 4 and provided with an agitation means 5 for the reaction medium by bubbling and/or by means of a mechanical member, a container 6 forming a secondary mixing chamber and whose capacity corresponds at least to the sum of the capacities of the reactors 3 of a module 2, being associated with each module 3 and a supplemental container 7 being provided, forming a principal mixing chamber whose capacity corresponds at least to the sum of the capacities of the different secondary mixing chambers 6, on the other hand by at least one circuit for the transfer of the contents of reactors 3 toward the container 6 associated with the synthesis module 2 in question and/or toward the container 7 and the controlled distribution of the contents of one or more containers 6 between the reactors 3 of the associated module 2 and/or of the content of the container 7 between the containers 6 or the reactors 3 of one, several or all of the modules 2 of the apparatus 1, as well as at least one supply or evacuation circuit connected particularly to the different inputs/outputs of the reactors 3 and of the containers 6, 7, these circuits permitting the circulation of fluid or fluids under the action of an inert or neutral propulsive gas and being formed by conduits or portions of conduits 8 interconnected with each other and connecting said reactors 3 and containers 6, 7 to each other and to reservoirs 9, 10, 11, 12, 13 for solutions and with expansion, suction and propulsive gas or bubbling injection lines, these connections being established temporarily by means of mono-path valves 15 or units of multi-path and monobloc valves 16, 17 constituting the programmable junctions of configuration of said circuits or control and management members of the inlets/outlets of the reactors 3 and containers 6, 7 and, finally, by a branched supervisory system 18 for management and monitoring of the circulation and of the distribution of the fluids in the mentioned circuits and of the temperature and agitation in the chambers 3″″ of the reactors 3, comprising particularly a computer unit 19 associated with electronic circuits 19′ for interfacing and multiplexing particularly for the control of the valves and valve units 15, 16, 17, of the heating means and cooling means 4, of the chambers 3″″ of the reactors 3 and, as the case may be, for the driving of the mechanical agitation members 5, and integrating interfaces 19″ of dialog with and programming by the user.

The conduits 8 of the different circulation circuits, whose passage cross-sections are suitable to the flow rates to be accommodated, define, accordingly, a plurality of possibilities of fluid connections, on the one hand, of the reactors 3 with the containers 6 and 7, on the other hand, of the containers 6 and 7 to each other, and, finally, of said reactors 3 and containers 6 and 7 with supply reservoirs 9 to 12, and recovery reservoirs 13, as the case may be by means of volumetric dosers 14, 14′. The valves 15 or valve units 16, 17 constitute active means of configuration and controlled activation of the circulation circuits or of portions of these latter, by opening or closing connection passages between the conduits or portions of conduits 8 pertaining to said valves or valve units and interconnected by these latter, by permitting or not access to the internal volume of a reactor 3 or of a container 6 or 7 and by permitting or not the pressurizing of reservoirs, reactors or container or containers by a propulsive gas ensuring injection and the circulation of the substances present in these receptacles, if desired in controlled quantities, in transfer paths previously defined by the connection of suitable conduits 8.

In the closed volume formed by the different closed receptacles mentioned above, in connection with different circulation circuits, the inert propulsive gas constitutes moreover a controlled atmosphere for the progress of the different reactions.

The liquid level present in the volumetric dosers 14 and 14′ is preferably predetermined by known corresponding opto-electronic devices whose output signals are transmitted to the computer unit 19 for evaluation, exploitation and possible consequent action.

Said opto-electronic devices could, as the case may be, deliver a simple signal in two conditions, corresponding to the exceeding or not of a certain predetermined level of liquid in the dosers 14 and 14′.

According to a preferred embodiment of the invention, shown in FIG. 3 of the accompanying drawings, said apparatus has a modular structure and is constituted by at least three sub-units 1′, 1″, namely a first sub-unit 1′ comprising a module 2 of five reactors 3, a secondary mixing chamber 6 and the principal mixing chamber 7 and at least two other sub-units 1″ of identical construction each comprising two modules 2 of five reactors 3 and their respectively associated secondary mixing chambers 6, each sub-unit 1′, 1″ comprising its own fluid circulation circuits, connecting its reactors 3 and containers 6, 7 together, to the reservoirs 9 to 13 and to associated 20 volumetric dosers 14, 14′, the reactors 3 and the secondary mixing chambers 6 of the two sub-units 1″ of the same construction being however connected in a fluid manner at least to the principal mixing chamber 7 forming a part of the first sub-unit 1′.

Those skilled in the art will understand that it is very easy, without major constructional modification of the structure described above, to add supplemental modular sub-units 1″ to increase the capacities of the apparatus 1.

This latter permits, as can be seen from the above description, mixing at two different levels, namely, at the level of a synthesis module 2 in relation to the container 6 associated with this latter, but also at the lever of all the synthesis modules 2, and/or of the different containers 6, which is to say at the level of the apparatus 1, in relation to the container 7, whose internal capacity will of course be adapted to the sum of the capacities of the reactors of the different modules.

As shown in FIG. 8 of the accompanying drawings, it is preferably provided that each sub-unit 1′, 1″ is associated with a branch 18′ for control, inspection and measurement of the supervisory system 18, these different branches 18′ being all connected to a series bus 18″ connected to the computer unit 19 controlling in particular the sequence of the different operative phases and whose transmission paths are multiplexed toward the numbers and means to be controlled and the detectors and measuring means of the different sub-units 1′, 1″ to open onto the outlet or inlet ports of the interface circuits 19′, these ports being arranged and grouped in branches 18′ with the image and as a function of the arrangement and of the physical or functional grouping of said members and means to control and said detectors and measuring means.

An extension of the machine 1 by addition of a supplemental sub-unit 1″ will accordingly translate to the simple addition of a supplemental branch 18′ corresponding to the level of the supervisory system 18.

The interfaces 19″ could for example, as shown also in FIG. 8 of the accompanying drawings, comprise a screen, a keyboard and a printer.

The screen will permit particularly visualizing in real time the current operations by reproducing, for example, the employed circuit and the circulation of the fluids in this circuit (see FIGS. 12, 13, 15, 17, 19).

According to one characteristic of the invention, shown particularly in FIGS. 3, 5 and 23 of the accompanying drawings, the reactors 3 of each module 4 are arranged among themselves equidistantly and equi-angularly, according to a circular configuration, and mounted in a support structure 20 carrying particularly also the valves and the multi-path valve units 15, 16a, 16b controlling the access to chambers 3″″ of the reactors 3 for the injection of substances and the extraction or evacuation of gas or substances to be recovered or to be eliminated, as well as if desired the valves or multi-path valve units 15, 16c controlling the access to said chambers 3″″ for emptying and withdrawing phasewise.

According to another characteristic of the invention, it is preferably provided that, for each reactor 3, the lower removable plug 3′″ ensures the sealing of a retention filter 21 for the resin serving as a synthesis support and comprises a passage 22 for controlled emptying of the liquids contained in said reactor 3 and if desired the injection of gas by bubbling and in that the upper plug 3″ comprises one or several passages 23 for injection of substances necessary to the synthesis and of various solvents and at least one channel 23′ for expansion and evacuation of the gases generated in the reaction 3 in question, each of said channels 23, 23′ being connected, at the level of its external opening, by a suitable branching connector 8′, to a corresponding opening of a channel or a portion of an outlet channel 16″, 17″ or inlet channel 16′, 17′ of at least one valve 15 or multi-path valve unit 16a, 16b for management of the inputs and outputs at the level of an upper plug 3″ belonging to the reactor 3 in question.

In addition to the communication channels for the upper and lower plugs, each reactor 3 could moreover be provided with one or several vertical conduits 23″ emptying into the internal volume forming the reaction chamber 3″″ slightly above the filter 21 forming retention means for the synthesis support, for example in the form of particles of resin.

In order to be able to ensure a precise temperature control and identical thermal conditions for the different reactors 3 of a same module 2, permitting simplification of the temperature regulation means and the measurement of this latter in a single reactor 3 per modular 2, the reactors 3 of each module 2 are thermally insulated from each other and relative to the external environment, the reactors 3 of a same module 2 being for example mounted in an insulating structure 20′ surrounding and insulating each of them.

Preferably, each reactor 3 is provided with heating and cooling means 4 for the lower part of its chamber 3″″ receiving the reaction medium and condensation means 4′ in the upper part of its chamber 3″″ to condense the vapors generated during heating of the reaction medium, said means 4, 4′ for heating/cooling and condensation, in the form of coils surrounding each reactor 3, mounted at the level of the portion of the chamber 3″″ of the reactor 3 to be regulated in temperature and acting through the material of the wall of the reactor 3 in question, and the insulating structure 20′ leaving the upper plugs 3″ and lower plugs 3′″ exposed and accessible, so as to be able to provide easy maintenance or replacement of these latter.

The reactors 3 and the modules 2 preferably correspond, as to their structure, their construction and their accessories, to the reactors and to the module described and shown in French patent application No. 00 03478, which is cited for this purpose in the present application.

Similarly, each secondary mixing container 6 and the principal mixing container 7 have bodies of tubular shape closed by an upper plug 6′, 7′ comprising one or several channels 25, 27 for injection of substances necessary for synthesis and solvents and at least one channel 25′, 27′ for expansion and evacuation of the gases, and to which belong the valves 15 and units 16a, 16b of multi-path valves for management of the inputs/outputs, and by a removable lower plug 6″, 7″ for emptying, provided with an evacuation passage 24, 26 whose diameter is controlled by a multi-path valve unit 16c and maintaining in sealed relation a retention filter 21′, 21″ for synthesis support, said containers 6 and 7 being moreover provided with a mechanical agitation member 5.

So as to be able also to carry out organic chemical reactions in the containers 6, after mixing the contents of the reactors 3 of the associated module 2, each container 6 forming a secondary mixing chamber can moreover be controlled as to temperature, at the level of its internal chamber, by being provided with thermal insulation 6′″ and by a means 4 for heating and cooling at least of the volume adapted to contain the reaction medium, associated with a cooling means 4′ for condensation of the vapors during heating of said reaction medium.

Such an arrangement also permits, when the apparatus 1 is used with parallel synthesis protocols, producing the same products of synthesis in said containers 6 as those that can be produced in the reactors 3, which greatly increases the total production volume of the machine 1.

Said containers 6 and 7 could thus have an identical structure and construction, apart from size, to those of the mentioned reactors 3.

However, the containers 7 forming a principal mixing chamber could also have a structure and a construction similar to that of the reactors described and shown in FR-A-2 664 602. Such a modified embodiment is also possible for the containers 6 forming secondary mixing chambers when no temperature monitoring is desired for these containers 6.

The valves 15 and the multi-path valve units 16, 17 provide programmable interconnections between the portions of the conduits 8 forming the different circulation circuits for corresponding liquid or gaseous fluids, according to a preferred embodiment of the invention, in terms of structure, construction and mode of operation, to the valves and to the multi-path distribution devices described and shown in FR-A-2 664 671 whose contents are incorporated in their totality, by reference, in the present application.

According to the functions they are to perform, the valve units 16, 17 will have different structures which can however be substantially classed in two different categories.

Thus, as shown for example in FIGS. 10 to 16 and 25 of the accompanying drawings, the multi-path valve units 16 of a first type each comprises a principal common channel 16′ for feeding or input, respectively collecting or output, which can be placed in fluid communication individually or group-wise, with a plurality of secondary outlet channels 16′″, respectively input channels 16″, separate during the actuation of one or several corresponding members for opening the passage or for lifting the blockage, controlled by the computer unit 19 of the supervisory system 18, said principal common channel 16′ being adapted, as the case may be, to be closed at the level of its end opening or openings by one or more similar members to permit the establishment of a passage communicating transversely between at least two secondary channels 16″, 16′″, the volumes of liquid transferred during opening of the communication passages being controlled by the computer unit 19 on the basis of the opening time and the pressure of the propulsive gas, for example nitrogen, applied to the reservoirs 9 to 13, to the volumetric dosers 14, 14′, to the reactors 3, to the secondary chambers 6 and/or to the principal chamber 7.

In the present description, the multi-path valve units 16 having the general structure described above are used for different functions, each of these uses being indicated by indices a to n.

Multi-path valve units 17 of a second type, shown particularly in FIGS. 10, 11 and 13 of the accompanying drawings, comprise a common principal channel 17′ that does not open to the exterior, connecting together a plurality of secondary channels 17″ opening, of which certain ones constitute input channels and the others constitute output channels, in a manner to form a selector permitting connecting one or several input channels 17″ to one or several output channels 17″, as a function of the actuation of or not of one or several passage opening members or the removal of the closure controlling each the passage between a secondary channel 17″ and the common principal channel 17.

In addition to the two types of valve units mentioned above, the apparatus 1 also comprises, as shown particularly in FIGS. 5 to 7, 10 to 13, 23 and 24 of the accompanying drawings, multi-path valve units 16a, 16b and 16c for control of the input/output at the level of the reactors 3 and of the containers 6 and 7. These valve units have two secondary input/output channels connected by a principal channel and a primary input/output channel, itself connected to the conduit, to the channel or to the input/output passage of the reactor 3 or of the container 6, 7 in question.

The multi-path valves 15 can themselves be in the form of independent valves, or be grouped in blocks of valves enclosing several valves 15 mounted in parallel (see FIG. 23).

The valves 15 can also be grouped in a radial distribution block 31, by being supplied in a common manner (see FIGS. 14 and 26).

Such a block 31 can for example correspond to that described and shown in the document DE-A-200 09 234 0 of the BURKERT company.

Finally, certain portions or certain branches of the circulation circuits are also defined by passive multi-valve connection junctions in the form of equi-molar dividers with an input path and four output paths 35 (see FIGS. 10 and 27) or with two output paths 8″ (see FIGS. 16 and 18). By reversing the branches of said dividers 35 and 8″, it is possible to provide collectors assembling several parallel flows into a single flow, conversely of the dividers.

Said dividers/collectors 35 and 8″ can, as the case may be, comprise a supplemental output for emptying or evacuation of the residues, for example opposite the input path.

Moreover, the input or output of said dividers or collectors could, as the case may be, be controlled by single-path valves (15), when no posterior control of the flow or flows is provided.

In FIGS. 5 to 7, 10, 11, 14, 16, 18, and 20 of the accompanying drawings, the single-path valves 15 or the individual valves of the multi-path valve units 16a, 16b and 16c for management of the inputs/outputs are represented in the form of a symbol constituted by a circle enclosing a cross or a multiplication sign.

As seen in FIG. 10 and by way of example for the sub-unit 1′, the apparatus 1 comprises for each module 2 a transfer/distribution/mixture circuit, this latter being principally constituted by a multi-path valve unit forming a selector 17 of which three secondary channels 17″ are respectively connected, on the one hand, to at least one transfer solution reservoir 12, on the other hand, to said container 6 forming a secondary mixing chamber for said module 2 and, finally, to the container 7 forming a principal mixing chamber, through a multi-path valve unit 16a for management of the inputs/outputs, at the level of the external openings of the vertical conduits or radial passages opening into the internal volume of said containers 6 and 7 slightly above the retention filter 21′, 21″ delimiting the bottom of the functional portion of said containers 6, 7, and of which a fourth secondary channel 17′, 17″ is connected to the input of the common channel 35′ of an equi-molar flow divider 35 whose outputs of the distribution channels 35″ are connected, by means of portions 35′″ of conduits having identical lengths and through valve units 16a for management of the inputs/outputs, to the different reactors 3 of the module 2 in question, at the level of the external openings of the conduits opening into the portions of the internal volumes forming reaction chambers 3′″ of said reactors 3 adapted to contain the reaction media, preferably slightly above the retention filters 21 delimiting the bottoms of said portions of volumes.

The container 7 is connected by a plurality of transfer lines 43 to the modules 2 of the sub-units 1″. These lines 43 each integrate a valve 15 controlling the circulation in the corresponding line 43 and can be connected to the container 7 at the level of the vertical conduits or of radial passages (in broken lines in FIG. 10).

FIG. 10 also shows a portion of a supply and recycling circuit for the transfer solution, extending to all the apparatus 1. This circuit is articulated about the principal reservoir of transfer solution 12, that can be placed under pressure by propulsive gas or expansion by actuation of suitable valves 15, and comprises a first valve unit 161 for monitoring of the distribution of 25 transfer solution to the different sub-units 1′, 1″ and a second multi-path valve unit 16m for monitoring the collection of the transfer solution from the different sub-units 1′, 1″, by means of connectors 35 connected to the outputs of the emptying paths of the valve units 16c for management of the inputs/outputs of the lower plugs 3′″, 6″ and 7″ of the reactors 3 and containers 6 and 7.

It will be noted that the flows from the outputs of the multi-path valve unit 161 are not directly carried to the secondary input channel 17″ of the selectors 17 in question of the different modules 2, but pass through a portion of a solvent distribution circuit, this so as to permit cleaning the transfer/distribution/mixing circuit by these solvents.

As shown in FIGS. 11 to 22 of the accompanying drawings, said apparatus 1 moreover comprises, for each module 2 or each sub-unit 1′, 1″, on the one hand, a circuit for supply and distribution of the combinative and secondary synthons and of the coupling reagents, and parallel segments of injection circuits for resin opening directly into the internal volume respectively of the chambers 3″″ of the reactors 3, of the secondary mixing chambers 6 and of the principal mixing chamber 7, on the other hand, a supply circuit for and of distribution of rinsing solvents, washing solvents and cleaning solvents, a supply circuit of and for distribution of general de-protection solvents, a supply circuit of and for distribution of TFA de-protection reagents and an expansion circuit for the different chambers 3″″, 6 and 7, connected to branching sites of the injection channels 23, 25, 27 or evacuation channels 23′, 25′, 27′ provided in the upper plug 3″, 6′, 7′ respectively of the reactors 3, containers forming secondary mixing chambers 6 and of the container forming a principal mixing chamber 7 and, finally, a circuit for monitored withdrawal of liquid phases present in said chambers and an emptying and evacuation circuit for waste, connected to branch sites formed at the outlet of the passages 22, 24, 26 provided in the lower plugs of the reactor 3, containers for secondary mixture 6 and of the principal mixing container 7.

According to one characteristic of the invention, shown more particularly in FIGS. 11 to 13 of the accompanying drawings, a distribution circuit for synthons and coupling reagents is associated with each sub-unit 1′, 1″ of said apparatus 1, this circuit being if desired constituted by two separate supply sub-circuits, namely, on the one hand, a supply sub-circuit for combinatory synthons comprising a unit 16d of multi-path valves for selecting synthons for each reactor 3 of a synthesis module 2, whose output of the common principal channel 16′ is connected to a first, of a pair mounted in cascade, of multi-path valve unit 16b for managing inputs/outputs of a passage or conduit opening into the internal volume of the corresponding reactor 3, preferably above a retention filter 21 for the solid synthesis support and, on the other hand, a supply sub-circuit for secondary synthons and coupling reagents comprising multi-path valve units 16e for selection of synthons and reagents, mounted in parallel, whose outputs of the principal channels 16′ are connected to the input channels of a selector 17, the output channels 17′″ of this latter being connected to the corresponding input channels 16″ of said first units 16b of multi-path valves for managing inputs/outputs at the level of the reactors 3 or with input channels 16″ of multi-path valve units 16b for management of inputs/outputs at the level of the passages or the conduits opening into the internal volumes of the associated containers 6, 7 of a module 2 or of the sub-unit 1 in question, as the case may be by means of a multi-path valve unit for multiplexed distribution 16f for the selection of the reactors 3 of the module 2 in question, the multi-path valve units for selection 16d and 16e each comprising moreover an input channel 16″ for the injection of washing and cleaning solvent or solvents, connected to the principal channel 16′ corresponding to the level of its end opposite the outlet, and the multi-path valve units for management 16b, selection 16f and forming a selector 17 each comprising an output channel 16′″ for the evacuation of waste toward reservoirs 13 for emptying and recovery by means of a collector 35, said output channel 16′″ being connected to the principal channel 16′ corresponding to the valve units 16b, 16f, 17 in question at the level of or adjacent one of its ends.

With the distribution circuit for synthons and coupling reagents described above is associated, as shown also in FIG. 11, a secondary circuit for selection and injection of solvents for washing and cleaning, comprising a multi-path valve unit 16n permitting the injection of different washing and cleaning liquids and of propulsive gas (N₂) in respective input channels 16″ of the multi-path valve units for selection of combinatory synthons 16d and for selection of secondary synthons and coupling reagents 16e.

It is thus possible to inject suitable cleaning solvents into all the mentioned distribution circuit, with evacuation toward the waste distributor at the level of the respective output channels of the second components of the multi-path valve units 16b.

The injection of solvents into the valve units 16d and 16e takes place at the level of input channel 16″ located opposite the output of the principal channel 16′ of each of said units.

The injection of such cleaning solvents could be carried out, for greater efficiency, discontinuously, with interposition of N₂ injection, so as to constitute alternating and sequential segments of liquid in gas.

In the pairs of multi-path valve units 16b for the management of the inputs/outputs of the reactors 3 and connected to each other by their common principal channels, the first unit permits carrying out a selection between the combinatory synthons, which are injected only into the reactors 3 and not into the containers 6 and 7, and the secondary synthons and reagents, the second unit comprising a first output channel connected to the input of the passage or conduit opening into the chamber 3″″ and a second output channel connected to the collector 35 (in FIG. 23 only the second unit is shown).

FIGS. 12 and 13 show (with symbols slightly different for the reactors and the containers) respectively, in a disjunctive manner, the sub-circuit for supply of combinatory synthons and the sub-circuit for supply of secondary synthons and coupling reagents. These figures could for example be displayed on a screen during distribution of one of the mentioned substances, showing the synthons, reagents or injected cleaning solvents, this to indicate to the user the operation taking place.

According to another characteristic of the invention, shown in FIGS. 14 and 15 of the accompanying drawings, a distribution circuit for washing, cleaning and rinsing solvents and transfer solution is associated with each sub-unit 1′, 1″, said circuit being principally constituted by at least one unit of multi-path valves for selection of solvents 16g whose input channels 16″ are connected to different reservoirs of solvent or solvents and of solution 10, 12 that can be separately pressurized by means of at least one unit 16h of multi-path valves for selectively placing under pressure, whose principal channel 16′ is supplied with propulsive gas and whose output channels 16′″ are each connected to a reservoir of solvent or solution 10, 12, the output of the principal channel 16′ of the valve unit for selection of solvents 16g being connected to the common supply input 31′ of a radial distributor block 31 comprising several multi-path valves 15 whose input channels 16″ are connected to said supply input 31′ and whose output channels 16′″ supply, through suitable volumetric dosers 14, 14′, on the one hand, an equi-molar flow divider 35 whose output channels 35″ are connected to a valve 15a for controlling the input of an injection channel 23 of which an upper plug 3″ of a reactor 3 of the modular 2 or of the sub-unit 1′ in question, on the other hand, the or a container 6 in question forming a secondary mixing chamber and, as the case may be, the container 7 forming a principal mixing chamber, this by means of an injection channel 25, 27 of their respective upper plug 6′, 7″, whose opening is controlled by a valve 15a.

When the number of solvents is great, there are preferably installed two selection multi-path valve units 16g in series, by connecting them to their respective principal channels 16′. A similar mounting could be carried out with multi-path valve units 16h for selective placing under pressure.

The capillaries for measuring the level of volumetric dosers 14 and 14′ are themselves connected, by a collector/divider 35 to the propulsive gas distribution circuit and to an expansion circuit by bubbling, under the control of two single-path valves 15.

According to another characteristic of the invention, and as shown in FIGS. 16 and 17 of the accompanying drawings, a distribution circuit for general de-protection reagents is associated with each sub-unit 1′, 1″, said circuit being principally constituted by at least two multi-path valve units 16i for selection of solvents, mounted in series, whose input channels 16″ are connected to different reservoirs 11 of general de-protection reagent or reagents that can be separately pressurized, preferably at different pressures, by means of at least two multi-path valve units 16j for pressurizing whose principal channel 16′ is supplied with propulsive gas and whose output channels 16″″ are each connected to a solvent reservoir 11, directly or by means of an intermediate expansion structure 32, the output of the principal channel 16′ of the multi-path valve unit 16i for selection of solvents being connected to the common supply input 31′ of a radial distributor block 31 comprising several multi-path valves whose input openings are connected to said common supply input 31′ and whose output openings supply, on the one hand, the input channel 35′ of a flow divider 35 whose output channels 35″ are each connected, by an input control valve 15b, to the external opening of an injection channel 23 of an upper plug 3″ of a reactor 3 of the module 2 or of the sub-unit 1′ in question and, on the other hand, the or a container 6 in question forming a secondary mixing chamber and, as the case may be, the container 7 forming a principal mixing chamber, this through an injection channel 25, 27 of their respective upper plug 6′, 7′, whose opening is controlled by a valve 15b.

Thus, each of the input channels 16″ of the two valve units 16i is pressurized with a specific propulsion gas provided by an output channel 16′″ corresponding to one of the two valve units 16j, by means of the reservoirs or cylinders of de-protection reagents 11 in question.

Moreover, a secondary circuit for distribution of coupling reagents is partially interconnected with each distribution circuit for de-protection reagents, the output of the principal channel 16′ of a multi-path valve unit 16k for selection of coupling reagents, whose input channels 16″ are connected to different reservoirs 9″ of coupling reagents, being also connected to the common supply input 31′ of the radial distributor 31 forming a part of the distribution circuit for general de-protection reagents, this if desired by means of a collector 35 forming a supply selector between the de-protection reagents and the coupling reagents.

The input channel 16″ of valve unit 16k, as well as the input channel 16″ of the first of the two valve units 16i, located at the ends of the principal channel 16′ respectively opposite their outputs, are directly connected to a propulsion gas supply line, for the purging of the circuit for distribution of de-protection reagents and of the secondary circuit for distribution of coupling reagents, the evacuation of the residues taking place through a supplemental output channel of one of the two diffusers 35, each connected by a corresponding evacuation line to a recovery receptacle 44 containing a neutralizing solution.

As shown in FIGS. 18 and 19 of the accompanying drawings, a distribution circuit for TFA de-protection reagents is provided for each sub-unit 1′, 1″, said circuit being essentially constituted by a radial distributor 31 whose supply channel 31′ is supplied in a controlled manner by means of a collector 8″ with solutions of TFA de-protection reagents at different concentrations drawn from separate reservoirs 11′ and whose output channels of the valves 15 are respectively connected, on the one hand, to a flow divider 35 whose output channels 35″ are each connected, via a valve 15c for input control, to an injection channel 23 of an upper plug 3″ of a reactor 3 of the or both modules 2 in question, on the other hand, to the injection channel or channels 25, 27 of the upper plugs 6′, 7′ of the container or containers 6, 7 in question forming a secondary or principal mixing chamber or chambers whose opening is controlled by a corresponding valve 15c.

The portions of conduits 35′″ connecting the radial channels 35 to the different valves 15c have identical lengths and the diffuser 35, as well as the distribution block 31, is connected by an evacuation line for the residues to a receptacle 45 containing a neutralizing solution. This pressurizing and expansion circuit for the two reservoirs 11′ of TFA de-protection reagents will also have an expansion line causing bubbling in said receptacle 45.

According to another characteristic of the invention, shown in FIG. 20 of the accompanying drawings, the expansion circuit is associated with each sub-unit 1′, 1″, said circuit being constituted by a plurality of parallel expansion lines 33 including non-return valves 33′, connected at their upstream end through valves 15d for the control of corresponding inputs to the evacuation channels 23′, 25′, 27′ of the upper plugs 3″, 6′, 7′ of the reactors 3 and containers 6, 7 in question, and connected at their opposite downstream ends in a bubbling mounting 34 in a decontaminant liquid contained in a receptacle 34′ subject to vapor removal by a hood.

Similarly, a waste evacuation circuit from the reactors 3 and containers 6, 7, and as the case may be for withdrawal by their lower plug 3′″, 6″, 7″, is provided for each sub-unit 1′, 1″, the different evacuation lines being connected, on the one hand, by corresponding valves of the valve units 16c for control of the inputs/outputs, to output openings of the passages 22, 24, 26 of the lower plugs 3′″, 6″, 7″ of said reactors 3 and containers 6, 7 and, on the other hand, to input channels of waste collectors 8″, if desired mounted in cascade by interconnection and connected, as the case may be, by an output selector 17, to the emptying and recovery reservoirs 13.

As shown in FIG. 21 of the accompanying drawings, the circuit for distribution and injection of propulsion gas is principally constituted by a principal supply line 36 connected to a source 36′ of propulsive gas under high pressure and by several parallel secondary supply lines 37 branched from said principal supply line through expansion valves 38 with calibrated pressures, connected respectively, directly, to the reservoirs 9, 9′, 9″, 10, 11, 12 for solvents, reagents and basic substances for syntheses, and, indirectly, to the reactors 3 and to the containers 6, 7 forming principal and secondary mixing chambers, by different circuits for circulation of fluids, and each associated with a safety valve for overpressure 37′ mounted in parallel in the line 37 in question, each secondary supply line 37 including at least one dewatering module 39 with silica gel and a paper filter module 40 through which the propulsive gas flow circulates in said secondary line 37.

So as to ensure precise regulation of temperature for each assembly of module 2/container 6, each sub-unit 1′, 1″ of said apparatus 1 comprises a device for temperature regulation including a command and control unit 28, controlled by the computer unit 19 and forming in cooperation with probes 29 for measuring the internal temperature in the reactor chambers 3 and secondary mixing chambers 6 and transfer tubes 30 of the Dewar type, connected, on the one hand, to a source of fluid controlled as to temperature and, on the other hand, to the inputs of the heating/cooling means 4, as many independent loops for regulation and control of temperature, namely one for each of the modules 2 of reactors 3 and one for each of the secondary mixing chambers 6 (see FIG. 9).

More precisely, each synthesis module 2 is provided with a temperature regulation device in the form of a supply line 41 of the heating/cooling means 4 of the reactors 3, in the form of coils, with thermo-regulated gaseous fluid, comprising essentially a transfer tube of the Dewar type including a heating means, connected at one of its ends to said coils 4 and by its opposite end to a source 42 of gaseous fluid at a temperature substantially lower than the lowest temperature desired for the reactors 3, at least one, and preferably several, of the reactors 3 including a measuring probe 29 for the temperature in their internal volume whose output signal is evaluated by a command and control unit 28 for the gaseous flow rate and of its heating in the transfer tube 30, forming with said probe or probes 29 a temperature regulation loop of each module 2 in question, said command and control unit 28 being if desired common to at least two modules 2 or to a sub-unit 1′, 1″.

Each reactor 3 and secondary container 6 comprises, moreover, as shown in FIG. 4, a condensation means 4′ permitting, in association with the expansion circuit shown in FIG. 20, controlling the reflux in said reactors 3 and containers 6.

The condensation means 4′, for example in the form of coils surrounding the reactor bodies or containers to be regulated (see also French patent application No. 00/03478 mentioned above), could be supplied with water at ambient temperature whose flow rate is regulated as a function of the solvent in question (dew point).

This regulation is carried out with a visual control at the level of the non-return valves 33′ (having transparent bodies and mounted in a visible manner on the apparatus), with verification of the presence or not of condensation in these latter, and/or in the receptacle 34′ (appearance of bubbles).

Similarly, these valves 33′ permit avoiding possible suction of decontaminant liquid into the reactors 3 or containers 6 when these latter are subjected to cooling giving rise to vacuum in their respective chambers.

Given the construction and symmetrical isolation of the modules 2, as well as the heating/cooling means 4 and the condensation means 4′, a single probe 29 for each module 2 will suffice, the reactors 3 being all subject to identical thermal conditions.

FIG. 22 shows schematically the resin injection lines, serving for synthesis support, into the reactors 3 and the containers 6, 7 by means of channels or passages opening slightly above the respective retention filters 21, 21′ and 21″.

Based on FIGS. 29, 30 and 31, there will be given hereafter the description of an illustrative example, which is not limiting, of a possible practical embodiment of a sub-unit 1″.

As shown in these figures, such a sub-unit 1″ could have, in addition to a functional construction symmetrical about the two assemblies of module 2/container 6, also a structure that is staged or by levels.

Thus, a lower store 46 receiving the reservoirs 10, 11, 12 of solvents, of general de-protection reagents, of TFA de-protection reagents and of recycled transfer solution, is installed at the level 0.

At level 1, there are inputs/outputs of the supply lines of the heating/cooling means 4 and of the condensation means 4′.

The container 6 and the reactors 3 mounted in modules 2 are installed at level 2 and the level immediately above (level 3) encloses valves 15a to 15d and valve units 16 and 16b controlling the inputs/outputs at the level of the reactors 3 and containers 6, as well as the motors and drive mechanisms for the agitating members 5.

Said level 3 also comprises, in the front position shown, the non-return valves 33′ and the receptacle 34 for expansion.

Level 4 includes the unit 28 for command and control regulating the temperature (from −80° C. to +100° C.) in the chambers of the reactors 3 and containers 6 of the sub-unit 1′ in question, as well as the electronic supervisor 28 for management and control of said sub-unit 1″.

The upper level (level 5) comprises itself a store 47 enclosing particularly the reservoirs 9, 9′ and 9″ of combinatory synthons, of secondary synthons and coupling reagents (with a staged arrangement), the volume meters 14, 14′ of the supplemental reservoirs 10 of cleaning, washing and rinsing solvents and the reservoir 12′ for pure transfer solution. Moreover, this store 47 could also enclose the electronics and the fluid control necessary to ensure the controlled distribution of the mentioned substances.

The different components, receptacles and portions mentioned are of course mounted or disposed on suitable supports, as the case may be removably, these supports being themselves installed on a load-bearing superstructure.

The present invention also has for its object a process for synthesis of organic molecules by means of apparatus 1, as described above, by use of a combinatory synthesis protocol in solid phase.

This process is characterized in that it comprises particularly at least one operation of transfer and mixing of the intermediate synthesis products on their solid synthesis support (synthesis products plus resin) present in the different reactors 3 of the different modules 2, in the container 7 forming a principal mixing chamber or in the containers 6 forming secondary mixing chambers and associated respectively each with a module 2, followed by an inverse operation of controlled transfer and distribution of the intermediate synthesis products on solid support, present in the containers 6 or in the container 7, either in the reactors 3 of the different modules 2 or the reactors 3 of the respective modules 2 in question, or in the different containers 6, said intermediate synthesis products on solid support being, before each transfer operation, suspended in a transfer solution that is chemically inert relative to said intermediate synthesis products, the volume of transfer solution being about ten times greater than the volume of intermediate synthesis products on solid support to be placed in suspension and said transfer solution being recovered and recycled after each operation and inverse operation mentioned above and before the performance of any consecutive operative phase (see also FIG. 10).

More precisely, each transfer and mixing operation consists essentially, for a given module 2 and its transfer/mixing/distribution circuit, in filling conduit portions 8, 8′ and 35′″ and the selector channels 17 and the divider 35, with fresh transfer solution, by placing under pressure a corresponding reservoir 12′ and by opening, in a repetitive manner, sequentially the corresponding valves of the valve units 16a of the different reactors 3 of the module 2 in question, then the corresponding valves of the valve unit 16a of the container 6 and/or 7, and then filling said portions of conduits and said channels by injection of the transfer solution into the reactors 3 and respective container or containers 6, 7, and in emptying said reactors 3 and containers 6 and/or 7 of their surplus transfer solution into the waste reservoir 13, then in filling said reactors 3 with transfer solution and actuating the mechanical agitation members 5 to place in suspension the intermediate synthesis products on solid support in the transfer liquid present in said reactors 3, in transferring the majority, preferably about 80%, of the content of said reactors 3 into the container 6 or 7, on the one hand, by opening the valves 15 or valve units 16a for management of the inputs/outputs, associated each with the external opening of a vertical conduit or a radial passage opening into the internal volume of the container 6 in question or of the container 7 and, on the other hand, by opening repetitively and sequentially the respective valves of the valve units 16a for management of the inputs/outputs of the different reactors 3 of the module 2 in question, connected to the external openings of conduits opening into the reaction chambers 3″″ slightly above the retention filters 21, this by placing successively said chambers 3″″ under the pressure of propulsive gas during given time intervals and after having configured in a suitable way the selector 17 associated with the module 2 in question, and then evacuating the rest of the transfer solution through emptying passages 22 of the lower plugs 3′″ of said reactors 3 by flowing into the reservoir 12, by filling said reactors 3 again with transfer solution from the reservoir 12 placed under pressure and in agitating their resulting contents, in repeating the sequential transfer and evacuation operations mentioned above and, finally, in repeating at least a third time said mentioned operations of filling the reactors, agitation, sequential transfer and evacuation.

When the operation of transfer/mixing concerns the container 7 and several, or even all of the modules 2, the different operative phases described above and involving the reactors 3 could be executed simultaneously at the level of the different modules 2 and sub-units 1′, 1″ in question.

Similarly, each transfer and controlled distribution operation consists essentially, after washing portions of conduits 8, 35′″ and selector channels 17 and the, divider 35 adapted to form by co-action the desired transfer and distribution circuit, in placing in suspension the intermediate synthesis products, and as the case may be the associated synthesis support, by injection of transfer solution from a corresponding reservoir 12 into the container 7 or the container or containers 6 in question and consecutive agitation by means of a mechanical agitation member 5, in filling the transfer and distribution circuit with transfer solution, in opening the valve or valves 15 or valve unit or units 16a for managing the inputs and outputs associated each with the external opening of a vertical conduit or a radial passage opening into the internal volume of the or each of said container 6 or container 7 in question, in configuring the selector or selectors 17 associated with the module or modules 2 in question so as to establish communication with the divider or dividers 15 of said module or modules 2, in placing under pressure of propulsive gas the container 6 or the container or containers 7, then in actuating the opening, sequentially and consecutively, according to cycles in loops, the respective valves of the valve units 16a for management of the inputs/outputs of the different reactors 3 of the module or modules 2 in question controlling access at the level of the external openings of conduits opening into the reaction chambers 3″″ of said reactors 3, as the case may be simultaneously for reactors 3 of different modules 2, in repeating the mentioned cycles a number of times sufficient to reduce substantially the volume of solution in the container or containers 6 or 7, preferably by about 75 to 95%, then by injecting an additional quantity of transfer solution into the container or containers 6 or 7 and in agitating the resulting mixture, in transferring said mixture as precedingly from the container or containers 6 or 7 to the reactors 3 in question, in repeating these two last operative phases at least one more time, then in emptying the container or containers 6 or 7 in question and withdrawing the transfer solution from said reactors 3 and in recycling by return movement toward the corresponding reservoir 12.

According to one characteristic of the invention, the division of fractionation by distribution, between the different reactors 3 of each module 2 in question, of the content of the container 7 or of the container 6 associated with said module 2 during a controlled transfer and distribution operation, is determined by control of the duration of actuation of the opening of the respective valves of the valve units 16a for management of the different reactors 3 of said module 2 during each cycle of actuation.

So as to avoid unequal or uncontrolled distribution of the intermediate synthesis products because of a poor determination of the beginning of injection of these latter, it is preferably provided that, during a first phase of the controlled transfer and distribution operation, corresponding to the evacuation of the pure transfer solution, which is to say not loaded with intermediate synthesis products, present in portions of circuit 8, 35′″ by successive fragmentary injections into the different reactors 3 or of the module or modules 2 in question, the duration of actuation of the valves of the valve units 16a for management of the inputs/outputs of said reactors 3 are identical for all the reactors 3 and of short length, in particular at the end of said evacuation phase and the beginning of the injection phase into the reactors 3 of transfer solution loaded with intermediate synthesis products from the container or containers 6, 7.

So as to reduce the duration of the transfer/distribution phases, it can also be provided that, in the case of a controlled transfer and distribution operation from the container 7 toward several modules 2, the reactors 3 of the same row of the different modules 2 in question are, in a repetitive manner and as a function of the actuation cycles, actuated simultaneously and for identical periods of time in the case of an equi-molar distribution between modules 2.

According to a preferred embodiment of the invention, the transfer solution consists of a mixture of DCM (dichloromethane) and DMF (dimethylformamide), preferably with a mutual volume ratio of about 1.

It will be noted that the distribution of the contents of a container 6 between the reactors 3 of the module 2 associated with this latter, and the distribution of the content of the container 7 between the reactors 3 of the different modules 2, or between the different containers 6, can be carried out in a manner of equality (same quantities injected into the different reactors 3 or containers 6) or without equality, by controlling the distributed quantities by means of actuating time of the valves or valve units in question by these transfers.

There should also be emphasized here the very great precision, both in quantitative and in qualitative terms, of the transfer/distribution operation, particularly by using actuating sequences of the valves in question of short duration.

Thus, no matter what the distribution of the substances in the containers 6 or the container 7, the transfer/distribution operation according to the invention permits having exact images, by duplication, of the mixture present in this or these containers 6 or 7 in the target reactors 3, removal of substance in this container or containers 6 or 7 taking place by successive strata of small thickness in which the mixture can be considered as being substantially homogeneous, these strata being then alternatively and regularly transferred to the different reactors 3 in question.

Moreover, the agitation of the contents of the containers 6 and 7 before each transfer/distribution operation permits both de-agglomerating if desired the substances present and homogenizing their distribution.

These operations of transfer/mixing and of transfer, although described in connection with the automatic synthesis apparatus 1 according to the invention and within the scope of a synthesis process for organic materials, can of course equally well be used in other contexts than that of the present invention and in other applications requiring the transfer of liquid substances between several primary containers (reactors 3 for example) and a secondary container (container 6 or 7) whose capacity is sufficient to accept the contents of said primary containers, in particular when it is necessary to provide a homogeneous and precisely balanced distribution of the content of the secondary container into the different primary containers in question.

Thus, it suffices for the practice of these operations to have control means for the evacuation and admission of substances in the primary containers and in the secondary container, that are able to be controlled precisely, according to the operative scheme described above, and of a circuit of equivalent transfer lines for the different primary containers (same length, same cross section, same division ratio).

Of course, the invention is not limited to the embodiment described and shown in the accompanying drawings. Modifications remain possible, particularly as to the construction of the various elements or by substitution of technical equivalents, without thereby departing from the scope of protection of the invention.

1. Automatic apparatus for the synthesis of organic molecules, particularly in solid phase, according to combinative or parallel synthesis protocols, characterized in that it is principally constituted, on the one hand, by several synthesis modules (2) each comprising between three and ten, preferably five, reactors (3) each formed by a tubular body (3′) delimiting, in cooperation with an upper injection and expansion plug (3″) and a lower removable plug (3′″) for controlled withdrawal and emptying, a reaction chamber (3″″) normally closed and sealed and controlled as to temperature by heating and cooling means (4) and provided with agitation means (5) of the reaction medium by bubbling and/or by means of a mechanical member, a container (6) forming a secondary mixing chamber and whose capacity corresponds at least to the sum of the capacities of the reactors (3) of a module (2), being associated with each module (3) and a supplementary container (7) being provided, forming a principal mixing chamber whose capacity corresponds at least to the sum of the capacities of the different secondary mixing chambers (6), on the other hand by at least one circuit for the transfer of the contents of the reactors (3) toward the container (6) associated with the synthesis module (2) in question and/or toward the container (7) and the controlled distribution of the content of one or more of the containers (6) between the reactors (3) of the associated module (2) and/or of the content of the container (7) between the containers (6) or the reactors (3) of one or several or all of the modules (2) of the apparatus (1), as well as at least one supply or evacuation circuit connected particularly to the different inputs/outputs of the reactors (3) and of the containers (6, 7), these circuits permitting the circulation of the fluid or fluids under the action of an inert or neutral propulsion gas and being formed by conduits or portions of conduits (8) interconnected with each other and connecting said reactors (3) and containers (6, 7) together and to reservoirs (9, 10, 11, 12, 13) of solutions and to expansion, suction and propulsion or bubbling gas injection lines, these connections being established temporarily by means of mono-path valves (15) or units (16, 17) of multi-path and monobloc valves constituting programmable junctions of configuration of said circuits or of control and management members for the inputs/outputs of the reactors (3) and containers (6, 7) and, finally, by a branched supervisor (18) for management and control of the circulation and of the distribution of the fluids in the mentioned circuits and of the temperature and agitation in the chambers (3″″) of the reactors (3), comprising particularly a computer unit (19) associated with electronic circuits (19′) for interfacing and multiplexing particularly for the control of the valves and valve units (15, 16, 17), of the heating and cooling means (4) of the chambers (3″″) of the reactors (3) and, as the case may be, the driving of the mechanical agitation members (5), and integrating dialog interfaces (19″) with and of programming by the user.

2. Apparatus according to claim 1, characterized in that it has a modular structure and is constituted by at least three sub-units (1′, 1″), namely a first sub-unit (1′) comprising a module (2) of five reactors (3), a secondary mixing chamber (6) and the principal mixing chamber (7) and at least two other sub-units (1″) of identical constructions comprising each two modules (2) of five reactors (3) and their secondary mixing chambers (6) respectively associated therewith, each sub-unit (1′, 1″) comprising its own fluid circulation circuits, connecting its reactors (3) and containers (6, 7) to each other, to the reservoirs (9 to 13) and to associated volumetric dosers (14, 14′), the reactors (3) and the secondary mixing chambers (6) of two sub-units (1″) of the same construction being nevertheless being connected, in a fluid manner, at least to the principal mixing chamber (7) forming a portion of the first sub-unit (1′).

3. Apparatus according to claim 2, characterized in that with each sub-unit (1′, 1″) is associated a control branch (18′), for control and measurement of the supervisor system (18), these different branches (18′) being all connected to a series bus (18″) connected to the computer unit (19) controlling in particular the progress of the different operative phases and whose transmission paths are multiplexed toward the numbers and means to be controlled and the detectors and measurement means for the different sub-units (1′, 1″) to open on outlet or inlet ports of the interface circuits (19′), these ports being arranged and grouped in branches (18′) with the image and as a function of the arrangement and of the physical or functional grouping of said members and means to be controlled and said detectors and measurement means.

4. Apparatus according to claim 1, characterized in that the reactors (3) of each module (2) are arranged between themselves in an equidistant and equiangular manner, according to a circular configuration and mounted in a support structure (20) carrying particularly also the valves and the multi-path valve units (15, 16a, 16b) controlling the access to the chambers (3″″) of the reactors (3) for the injection of substances and the extraction or evacuation of gas or substances to be recovered or eliminated, as well as if desired the valves or multi-path valve unit (15, 16c) controlling the access to said chambers (3″″) for emptying and withdrawing by phases.

5. Apparatus according to claim 1, characterized in that, for each reactor (3), the lower removable plug (3″″) ensures the sealing of a retention filter (21) of the resin serving as a synthesis support and comprises a passage (22) for controlled emptying of the liquids contained in said reactor (3) and if desired the injection of a bubbling gas and in that the upper plug (3″) comprises one or several channels (23) for the injection of substances necessary for the synthesis and of various solvents and at least one channel (23′) for expansion and evacuation of the gases generated in the reactor (3) in question, each of said channels (23, 23′) being connected, at its external opening, with a suitable branching connection (8′), with a corresponding opening of a channel or a portion of outlet channel (16″, 17″) or input channel (16′, 17′) of at least one valve (15) or unit of multi-path valves (16a, 16b) for management of the inputs/outputs of an upper plug (3″) and belonging to the reactor (3) in question.

6. Apparatus according to claim 1, characterized in that the reactors (3) of each module (2) are thermally insulated from each other and from the external medium, the reactors (3) of a same module (2) being for example mounted in an insulating structure (20′) surrounding and isolating each one from the others, and in that each reactor (3) is provided with heating and cooling means (4) of the lower portion of its chamber (3″″) receiving the reaction medium and of a condensation means (4′) of the upper portion of its chamber (3″″) to condense vapors generated during heating of the reaction medium, said heating/cooling and condensation means (4, 4′), in the form of coils surrounding each reactor (3), mounted in the portion of the chamber (3″″) of the reactor (3) to be regulated as to temperature and acting through the material of the wall of the reactor in question (3), and the insulating structure (20′) leaving the upper and lower plugs (3″, 3′″) exposed and accessible.

7. Apparatus according to claim 2, characterized in that each secondary mixture container (6) and the principal mixture container (7) have bodies of tubular shape closed by an upper plug (6′, 7′) comprising one or several channels (25, 27) for the injection of substances necessary for the synthesis and of solvents and at least one channel (25′, 27′) for expansion and evacuation of gases, and with which are asssociated valves (15) and units (16a, 16b) of multi-path valves for management of the inputs/outputs, and by a lower removable emptying plug (6″, 7″), provided with an evacuation passage (24, 26) of which the outlet is controlled by a multi-path valve unit (16c) and maintaining sealed a retention filter (21′, 21″) for synthesis support, said containers (6 and 7) being moreover provided with a mechanical agitation member (5).

8. Apparatus according to claim 7, characterized in that each secondary mixture container (6) is moreover controlled as to temperature, in its internal chamber, by being provided with thermal insulation (6′″) and means (4) for heating and cooling at least of the volume adapted to contain the reaction medium, associated with a cooling means (4′) for the condensation of vapors generated during heating of said reaction medium.

9. Apparatus according to claim 1, characterized in that the multi-path valve units (16) of a first type each comprise a common principal channel (16′) for supply or input, respectively collector or outlet, being adapted to be placed in fluid communication, individually or group-wise, with a plurality of output secondary channels (16′″), respectively of input channels (16″), separate during actuation of one or several corresponding members for opening the passage or for raising the closure, controlled by the computer unit (19) of the supervisory system (18), said common principal channel (16′) being adapted, as the case may be, to be closed at its end opening or openings by one or more similar members to permit the establishment of a transverse communication passage between at least two secondary channels (16″, 16′″), the volumes of liquid transferred during opening of the communication passages being controlled by the computer unit (19) based on the opening time and the pressure of the propulsion gas, for example nitrogen, applied to the reservoirs (9 to 13), to the volumetric dosers (14, 14′), to the reactors (3), to the secondary chambers (6) and/or to the principal chamber (7).

10. Apparatus according to claim 9, characterized in that multi-path valve units (17) of a second type comprise a common principal channel (17′) that does not open to the outside, connecting together a plurality of opening secondary channels (17″) of which certain ones constitute input channels and of which the others constitute output channels, so as to form a selector permitting connecting one or several input channels (17″) to one or several output channels (17″), as a function of the actuation or inactivation of one or several members for opening a passage or for removing an obstruction controlling each one the passage between a secondary channel (17″) and the common principal channel (17).

11. Apparatus according to claim 1, characterized in that it comprises, for each module (2) a transfer/distribution/mixing circuit, this latter being principally constituted by a multi-path valve unit forming a selector (17) of which three secondary channels (17″) are respectively connected, on the one hand, to at least one reservoir of transfer solution (12), and on the other hand to said container (6) forming a secondary mixing chamber for said module (2) and, finally, to the container (7) forming a principal mixing chamber, via a multi-path valve unit (16a) for management of the inputs/outputs, of the external openings of vertical conduits or radial passages opening into the internal volume of said containers (6 and 7) slightly above the retention filter (21′, 21″) delimiting the bottom of the functional portion of said containers (6, 7), and of which a fourth secondary channel (17′, 17″) is connected to the input of the common channel (35′) of an equi-molar flow divider (35) whose outputs of the distribution channels (35″) are connected, by means of portions (35′″) of conduits having identical lengths and via valve units (16a) for management of the inputs/outputs, to the different reactors (3) of the module (2) in question, at external openings of conduits opening into the portions of the internal volumes forming reaction chambers (3′″) of said reactors (3) adapted to contain the reaction media, preferably slightly above filtration filters (21) limiting the bottoms of said portions of volumes.

12. Apparatus according to claim 5, characterized in that it comprises, for each module (2) or each sub-unit (1′, 1″), on the one hand, a supply circuit for and of distribution of combinatory and secondary synthons and coupling reagents, and parallel segments of injection circuits for resin opening directly into the internal volume respectively of the chambers (3″″) of reactors (3), of the secondary mixing chambers (6) and of the principal mixing chamber (7), on the other hand, a supply circuit of and for distribution of rinsing, washing cleaning solvents, a supply circuit for and of distribution of general de-protection reagents, a supply circuit of and for distribution of TFA de-protection reagents and an expansion circuit for the different chambers (3″″, 6 and 7), connected to branches of injection channels (23, 25, 27) or evacuation channels (23′, 25′, 27′) provided in the respective upper plug (3″, 6′, 7′) of the reactors (3), of the containers forming secondary mixing chambers (6) and of the container forming the principal mixing chamber (7) and, finally, a controlled withdrawal circuit of liquid phases present in said chambers and a circuit for emptying and evacuation of waste, connected to branch sites formed at the outlet of passages (24, 26, 28) provided in the lower plugs of the reactors (3), of the secondary mixing containers (6) and of the principal mixing container (7).

13. Apparatus according to claim 7, characterized in that a distribution circuit of synthons and coupling reagents is associated with each sub-unit (1′, 1″) of said apparatus (1), this circuit being if desired constituted by two separate supply sub-circuits, namely, on the one hand, a supply sub-circuit for combinatory synthons comprising a unit (16d) of multi-path valves for selection of synthons for each reactor (3) of a synthesis module (2), whose output of the common principal channel (16′) is connected to a first of a pair mounted in cascade of units (16b) of multi-path valves for the management of inputs/outputs of a passage or conduit opening into the internal volume of the corresponding reactor (3), preferably above a retention filter (21) for the solid synthesis support and, on the other hand, a sub-circuit for supplying with secondary synthons and with coupling reagents comprising units (16e) of multi-path valves for selection of synthons and of reagents, mounted in parallel, whose outputs of the principal channels (16′) are connected to the input channels of a selector (17), the output channels (17′″) of this latter being connected to the corresponding input channels (16″) of said primary units (16b) of multi-path valves for management of inputs/outputs in the reactors (3) or with input channels (16″) of multi-path valve units (16b) for management of the inputs/outputs of the passages or of conduits opening into the internal volumes of the containers (6, 7) in question of a module (2) or of the sub-unit (1) in question, as the case may be by means of a multi-path valve unit for multiplexed distribution (16f) for the selection of the reactors (3) of the module (2) in question, the multi-path valve units for selection (16d and 16e) each comprising moreover an input channel (16″) for the injection of washing and cleaning solvent or solvents, connected to the corresponding principal channel (16′) at its end opposite its output, and the units of multi-path management valves (16b), of selection units (16f) and forming a selector (17) each comprising an output channel (16′″) for the evacuation of waste toward reservoirs (13) for emptying and recovery by means of a collector (35), said output channel (16′″) being connected to the corresponding principal channel (16′) of the valve unit (16b, 16f, 17) in question at or adjacent one of its ends.

14. Apparatus according to claim 7, characterized in that a distribution circuit for solvents for washing and cleaning and rinsing and for transfer solution is associated with each sub-unit (1′, 1″), said circuit being principally constituted by at least one unit of multi-path valves for selection of solvents (16g) whose input channels (16″) are connected to different solvent and solution reservoirs (10, 12) that can be placed under pressure separately by means of at least one unit (16h) of multi-path valves for placing under pressure selectively, whose principal channel (16′) is supplied with propulsive gas and whose output channels (16′″) are each connected to a reservoir of solvent or solution (10, 12), the output of the principal channel (16′) of the valve unit for selection of solvents (16g) being connected to the common supply input (31′) of a radial distributor block (31) comprising several mono-path valves (15) whose input channels (16″) are connected to said supply input (31′) and whose output channels (16′″) supply, via suitable volumetric dosers (14, 14′), on the one hand an equi-molar flow divider (35) of which the output channels (35′) are connected to a valve (15a) for the control of the input of an injection channel (23) of an upper plug (3″) of a reactor (3) of the module (2) or of the sub-unit (1′) in question, on the other hand, the or a container (6) in question forming a secondary mixing chamber and, as the case may be, the container (7) forming the principal mixing chamber, this by means of an injection channel (25, 27) of their respective upper plug (6′, 7″), of which the opening is controlled by a valve (15a).

15. Apparatus according to claim 7, characterized in that a distribution circuit for general de-protection reagents is associated with each sub-unit (1′, 1″), said circuit being principally constituted by at least two units (16i) of multi-path valves for selection of solvents, mounted in series, whose input channels (16″) are connected to different reservoirs (11) of general de-protection reagent or reagents that can be separately placed under pressure, preferably at different pressures, by means of at least two units (16j) of multi-path valves for placing under pressure whose principal channel (16′) is supplied with propulsive gas and whose output channels (16″″) are each connected to a reservoir (11) of solvents, directly or by means of an intermediate expansion structure (32), the output of the principal channel (16′) of the unit (16i) of multi-path valves for selection of solvents being connected to the common supply input (31′) of a radial distributor block (31) comprising several mono-path valves (15) whose input openings are connected to said common supply input (31′) and whose output openings supply, on the one hand, the input channel (35′) of a flow divider (35) whose output channels (35″) are each connected, via a valve (15b) for input control, to the external opening of an injection channel (23) of an upper plug (3″) of a reactor (3) of the module (2) or of the sub-unit (1′) in question and, on the other hand, the or one container (6) in question forming the secondary mixing chamber and, as the case may be, the container (7) forming the principal mixing chamber, this via an injection channel (25, 27) of their respective upper plug (6′, 7′), whose opening is controlled by a valve (15b).

16. Apparatus according to claim 15, characterized in that a secondary distribution circuit for coupling reagents is partially interconnected with each distribution circuit for de-protection reagents, the output of the principal channel (16′) of a unit (16k) of multi-path valves for selection of coupling reagents, whose input channels (16″) are connected to different reservoirs (9″) of coupling reagents, being also connected to the common supply input (31′) of the radial distributor (31) forming a portion of the distribution circuit for general de-protection reagents, this if desired by means of a collector (35) forming a supply selector between the de-protection reagents and the coupling reagents.

17. Apparatus according to claim 7, characterized in that a distribution circuit for TFA de-protection reagents is provided for each sub-unit (1′, 1″), said circuit being essentially constituted by a radial distributor (31) whose supply channel (31′) is supplied in a controlled manner by means of a collector (8″) with TFA de-protection reagents at different concentration from separate reservoirs (11′) and whose output channels of the valves (15) are respectively connected, on the one hand, to a flow divider (35) whose output channels (35″) are each connected, via a valve (15c) for input control, to an injection channel (23) of an upper plug (3″) of a reactor (3) of the one or two modules (2) in question, on the other hand to the injection channels (25, 27) of the upper plugs (6′, 7′) of the container or containers (6, 7) in question forming secondary or principal mixing chambers, whose opening is controlled by a corresponding valve (15c).

18. Apparatus according to claim 7, characterized in that an expansion circuit is associated with each sub-unit (1′, 1″), said circuit being constituted by a plurality of parallel expansion lines (33) including non-return valves (33′), connected at their upstream end via valves (15d) for input control corresponding to the evacuation channels (23′, 25′, 27′) of the upper plugs (3″, 6′, 7′) of the reactors (3) and associated containers (6, 7) and connected at their opposite downstream ends in a bubbling mounting in a decontaminant liquid (34) contained in a receptacle (34′) subjected to the suction of a hood, and in that an evacuation circuit for waste from the reactors (3) and containers (6, 7), and as the case may be for withdrawal by their lower plug (3′″, 6″, 7″), is provided for each sub-unit (1′, 1″), the different evacuation lines being connected, on the one hand, via corresponding valves of the valve units (16c) for management of the inputs/outputs, to the outlet openings of the passages (22, 24, 26) of the lower plugs (3′″, 6″, 7″) of said reactors (3) and containers (6, 7) and, on the other hand, to input channels of collectors (8″) of waste, if desired mounted in cascade by interconnection and connected, as the case may be, by an outlet selector (17), to the emptying and recovery reservoirs (13).

19. Apparatus according to claim 12, characterized in that the circuit for distribution and injection of propulsive gas is principally constituted by a principal supply line (36) connected to a source (36) of propulsive gas under high pressure and by several parallel secondary supply lines (37) derived from said principal supply line via expansion valves (38) of calibrated pressures, connected respectively directly to the reservoirs (9, 9′, 9″, 10, 11, 12) for solvents, reagents and basic substances for syntheses, and, indirectly to the reactors (3) and containers (6, 7) forming principal and secondary mixing chambers, by the different circuits for circulation of fluids, and each associated with a safety valve (37′) for overpressure mounted in parallel in the line (37) in question, each secondary supply line (37) including at least one dewatering module (39) of silica gel and a paper filter module (40) traversed by the flow of propulsive gas circulating in said secondary line (37).

20. Apparatus according to claim 2, characterized in that each sub-unit (1′, 1″) of said apparatus (1) comprises a temperature regulating device including a unit (28) for command and control, controlled by the computer unit (19) and forming in cooperation with probes (29) for measuring internal temperature of the reaction chambers (3) and secondary mixing chambers (6) and transfer tubes (30) of the Dewar type connected, on the one hand, to a source of fluid controlled as to temperature and, on the other hand, to the inputs of the heating/cooling means (4), as many independent loops for regulation and control of temperature, namely one for each of the modules (2) of reactors (3) and one for each of the secondary mixing chambers (6).

21. Apparatus according to claim 11, characterized in that each synthesis module (2) is provided with a device for temperature regulation in the form of a supply line (41) of the heating/cooling means (4) of the reactors (3), in the form of coils, with thermo-regulated gaseous fluid, comprising essentially a transfer tube (30) of the Dewar type having heating means, connected by one of its ends to said coils (4) and by its opposite end to a source (42) of gaseous fluid at a temperature substantially below the lowest temperature desired for the reactors (3), at least one, and preferably several, of the reactors (3) having a measuring probe (29) of the temperature in their internal volume whose output signal is evaluated by a unit (28) for command and control of the flow of gaseous fluid and of its heating in the transfer tube (30), forming with said probe or probes (29) a regulation loop of the temperature of each module (2) in question, said command and control unit (28) being if desired common to at least two modules (2) or to a sub-unit (1′, 1″).

22. Process for the synthesis of organic molecules by means of apparatus according to claim 11, by application of a combinative synthesis protocol in solid phase, characterized in that it comprises particularly at least one operation of transfer and mixture of intermediate synthesis products with their solid synthesis support present in different reactors (3) of the different modules (2), in a container (7) forming a principal mixing chamber or in containers (6) forming secondary mixing chambers and associated respectively each with a module (2), followed by an inverse operation of transfer and controlled distribution of the intermediate synthesis products on solid support and, as the case may be, of the synthesis support, present in containers (6) or in the container (7), either in the reactors (3) of the different modules (2) or the reactors (3) of the modules (2) respectively in question, or in the different containers (6), said intermediate synthesis products on solid support being, before each transfer operation, placed in suspension in a transfer solution that is chemically inert relative to said intermediate synthesis products, the volume of transfer solution being about ten times greater than the volume of said intermediate synthesis products on solid support to be placed in suspension and said transfer solution being recovered and recycled after each operation and inverse operation mentioned above and before undertaking any consecutive operative phase.

23. Synthesis process according to claim 22, characterized in that each transfer operation and mixing operation consists essentially, for a given module (2) and its transfer/mixing/distribution circuit, in filling the portions of conduits (8, 8′) and (35′″) and the channels of the selector (17) and of the divider (35), with fresh transfer solution by placing under pressure a corresponding reservoir (12′) and by opening in a repetitive manner, sequentially, the corresponding valves of the valve units (16a) of the different reactors (3) of the module (2) in question, then the corresponding valves of the valve units (16a) of the container (6) and/or (7), in then filling said portions of conduits and said channels by injection of the transfer solution into the reactors (3) and container or containers (6 and/or 7) respectively, and in emptying said reactors (3) and containers (6 and/or 7) of their surplus of transfer solution and in actuating the mechanical agitation members (5) to place in suspension the intermediate synthesis products on solid support in the transfer liquid present in said reactors (3), in transferring the majority, preferably about 80%, of the content of said reactors (3) into the container (6 or 7), on the one hand, by opening the valves (15) or valve units (16a) for control of the inputs/outputs, associated each with the external opening of a vertical conduit or of a radial passage emptying into the internal volume of the container (6) in question or of the container (7) and, on the other hand, by opening in a repetitive and sequential manner the respective valves of the valve units (16a) for management of the inputs/outputs of the different reactors (3) of the module (2) in question, connected to the external openings of conduits opening into the reaction chambers (3″″) slightly above retention filters (21), this by placing successively said chambers (3″″) under pressure of propulsive gas during given time intervals and after having configured suitably the selector (17) associated with the module (2) in question, and then evacuating the rest of the transfer solution through emptying passages (22) of the lower plugs (3′″) of said reactors (3) by moving it into the reservoir (12), in filling said reactors (3) again with transfer solution from the reservoir (12) placed under pressure and in agitating the resulting content, in repeating the operations of sequential transfer and of evacuation mentioned above, and, finally, in repeating at least a third time said mentioned operations of filling the reactors (3), of agitation, of sequential transfer and of evacuation.

24. Synthesis process according to claim 22, characterized in that each operation of transfer and controlled distribution consists essentially, after washing portions of conduits (8, 35′″) and selector channels (17) and of the divider (35) adapted to form by co-action the desired transfer and distribution circuit, in placing in suspension the intermediate synthesis products, and as the case may be the associated synthesis support, by injection of transfer solution from a corresponding reservoir (12) into the container (7) or the container or containers (6) in question, and subsequent agitation by means of a mechanical agitation member (5), in filling the transfer and distribution circuit with transfer solution, in opening the valve or valves (15) of the valve unit or units (16a) for management of the inputs/outputs associated each with the external opening of a vertical conduit or of a radial passage opening into the internal volume of the or each of said containers (6) or of the container (7) in question, in configuring the selector or selectors (17) associated with the module or modules (2) in question so as to establish communication with the divider or dividers (15) of said module or modules (2), in placing under the pressure of propulsive gas the container (7) or the container or containers (6), then in actuating the opening, sequentially and consecutively, according to cycles in loops, of the respective valves of the valve units (16a) for management of the inputs/outputs of the different reactors (3) of the module or modules (2) in question controlling the access to the external openings of conduits opening into the reaction chambers (3″″) of said reactors (3), as the case may be simultaneously for the reactors (3) of different modules (2), in repeating the mentioned cycles a number of times sufficient substantially to reduce the volume of solution in the container or containers (6 or 7), preferably by about 75 to 95%, then in injecting an additional quantity of transfer solution into the container or containers (6 or 7) and in agitating the resulting mixture, in transferring said mixture as before from the container or containers (6 or 7) toward the reactors (3) in question, in repeating these last two operative phases at least once more, then emptying the container or containers (6 or 7) in question and withdrawing the transfer solution from said reactors (3) and in recycling it by bringing it back to the corresponding reservoir (12).

25. Synthesis process according to claim 24, characterized in that the fractionation by distribution, between the different reactors (3) of each module (2) in question, of the content of the container (7) or of the container (6) associated with said module (2) during a controlled distribution and transfer operation, is determined by the control of the durations of actuation of the opening of the respective valves of the valve units (16a) for managing different reactors (3) of said module (2) during each cycle of actuation.

26. Process according to claim 25, characterized in that during a first phase of the controlled transfer and distribution operation, corresponding to the evacuation of the pure transfer solution, which is to say not loaded with intermediate synthesis products, present in the circuit portions (8, 35′″) by successive fragmentary injections into the different reactors (3) of the module or modules (2) in question, the duration of actuation of the valves of the valve units (16a) for management of the inputs/outputs of said reactors (3) is identical for all the reactors (3) and of short length, in particular at the end of said evacuation phase and the beginning of the injection phase into the reactors (3) of transfer solution loaded with intermediate synthesis products from the container or containers (6, 7).

27. Synthesis process according to claim 24, characterized in that, in the case of a controlled transfer and distribution operation from a container (7) toward several modules (2), the reactors (3) of the same row of the different modules (2) in question are, in a repetitive manner and as a function of the actuation cycles, actuated simultaneously and for identical lengths of time in the case of an equi-molar distribution between modules (2).

28. Process according to claim 24, characterized in that the transfer solution consists of a mixture of DCM (dichloromethane) and DMF (dimethylformamide), preferably with a mutual volumetric ratio of about 1.
